(19) United States
(12) Patent Application Publication
(10) Pub. No.: US 2010/0190107 A1
(43) Pub. Date: Jul. 29, 2010

(54) CYCLIC COMPOUND, PHOTORESIST BASE MATERIAL AND PHOTORESIST COMPOSITION

(75) Inventors: Mitsuru Shibata, Chiba (JP); Takanori Owada, Chiba (JP); Akinori Yomogita, Chiba (JP); Takashi Kashiwamura, Chiba (JP); Masashi Sekikawa, Chiba (JP); Norio Tomotsu, Chiba (JP); Hirotoshi Ishii, Chiba (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Idemitsu Kosan Co. Ltd, Tokyo (JP)

(21) Appl. No.: 12/664,784

(22) PCT Filed: Jun. 13, 2008
(86) PCT No.: PCT/JP2008/060905

§ 371 Date: Dec. 15, 2009

(30) Foreign Application Priority Data
Jun. 15, 2007 (JP) 2007 158898
Nov. 7, 2007 (JP) 2007 289614

(51) Int. Cl. C07C 69/94; G03F 7/004; G03F 7/20; H01L 21/027

(52) U.S. Cl. 430/270.1; 560/59; 430/319

(57) ABSTRACT

A cyclic compound shown by the following formula (I):

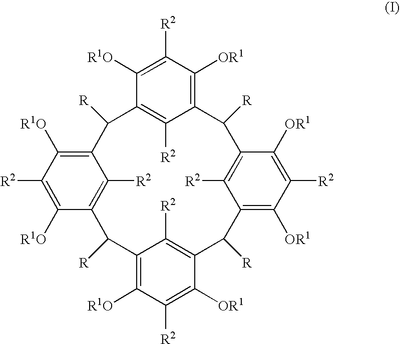

### TECHNICAL FIELD

The invention relates to a photoresist base material used in the fields of electricity and electronics such as a semiconductor, the optical field or other fields, in particular to a photoresist base material for ultrafine processing.

### BACKGROUND ART

Lithography by extreme ultraviolet light (hereinafter often referred to as “EUVL”) or by an electron beam is useful as a fine processing method with a high productivity and a high resolution in the production of a semiconductor or the like. A photoresist having a high sensitivity and a high resolution to be used in this lithography has been demanded. In respect of the productivity, resolution or the like of a desired fine pattern, it is indispensable to improve the sensitivity of a photoresist.

As the photoresist used in ultrafine processing by EUVL, for example, a chemically amplified polyhydroxy styrene-based photoresist which has been used in known ultrafine processing by means of a KrF laser can be given. It is known that this resist is capable of performing fine processing up to about 50 nm. However, if ultrafine processing by extreme ultraviolet light was conducted using this resist to produce a pattern finer than 50 nm, the production of which is the biggest advantage attained by processing by extreme ultraviolet light, although practicality was realized in respect of sensitivity and a resist outgas, it was impossible to attain the reduction of line edge roughness, which is most important. Therefore, it cannot be said that this resist fully brings out the performance instinct to extreme ultraviolet light. Under such circumstances, development of a photoresist which shows higher performance than ever has been required.

In view of the above-mentioned problems, for example, a method is proposed in which a chemically amplified positive type photoresist which has a higher concentration of a photo-acid generator than other resist compounds is used (for example, see Patent Document 1). However, as for a photoresist given as an example, which is formed of a base material composed of a terpolymer of hydroxystyrene/styrene/t-butyl acrylate, a photo-acid generator composed of di(t-butylphenyl)iodonium ortho-trifluoromethylsulfonate, which accounts for at least about 5 wt % of total solid matters, tetrabutylammonium hydroxide lactate and ethyl lactate, processing to a fineness up to 100 nm, which is exemplified as a case where an electron beam is used, is thought to be the limit. The main reason therefor is assumed to be as follows. The three-dimensional morphology of a mass of polymer compounds or each molecule of polymer compounds, which is used as the base material, is large. Such large three-dimensional morphology exerts adverse effects on the production line width and the surface roughness.

One of the inventors already proposed a calixresorcinarene compound as a photoresist material which has a high sensitivity and a high resolution (see Patent Documents 2 and 3). However, there has been a demand for a novel low-molecular organic compound which is amorphous at room temperature. At the same time, improvement of various performances such as etching resistance which often becomes important in semiconductor production processes has been required. In the current semiconductor production processes, since a photoresist base material is dissolved in a solvent for film formation, a photoresist base material is required to be highly soluble in a solvent for coating.

Patent Document 4 discloses a calixresorcinarene compound. Part of these compounds appear to be insufficient in solubility. In addition, the use thereof as a photoresist base material is not disclosed, and only an application in which these compounds are added as an additive to a photoresist base material composed of a known polymer is disclosed.

Patent Document 1: JP-A-2002-055457

Patent Document 2: JP-A-2004-191913

Patent Document 3: JP-A-2005-075767

Patent Document 4: U.S. Pat. No. 6,093,517

An object of the invention is to provide a compound and a composition which are suitable as a photoresist base material having characteristics such as high sensitivity, high resolution, high fine processability and improved solubility in a solvent for coating.

### DISCLOSURE OF THE INVENTION

The inventors have found that the above-mentioned problems are caused by the three-dimensional molecular morphology or the molecular structure of a photoresist base material composed of a polymer compound, or reactivity based on the structure of a protective group in the molecular structure thereof. Then, the inventors have found that a cyclic compound having a prescribed structure is useful as a photoresist base material, and led to the completion of the invention.

The invention provides the following cyclic compound or the like.

1. A cyclic compound shown by the following formula (I):

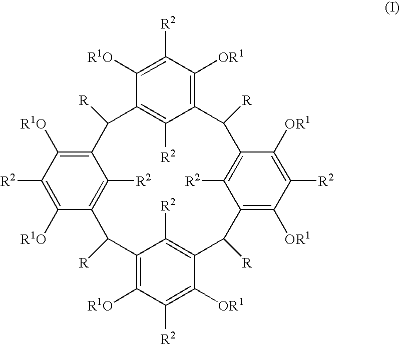

wherein R is a group shown by the following formula (1);

R¹s are independently hydrogen, a substituted or unsubstituted straight-chain aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group, or a group having a structure in which these groups and a divalent group (a substituted or unsubstituted alkylene group, a substituted or unsubstituted arylene group, a substituted or unsubstituted silylene group, a group formed by bonding two or more of these groups or a group formed by combining one or more of these groups and one or more of an ester group (—CO₂—), a carbonic ester group (—CO₃—) and an ether group (—O—)) are bonded;

R²s are independently hydrogen, a group shown by —O—R¹, a straight-chain aliphatic hydrocarbon group having 1 to 20 carbon atoms, a branched aliphatic hydrocarbon group having 3 to 12 carbon atoms, a cyclic aliphatic hydrocarbon group having 3 to 20 carbon atoms, an aromatic group having 6 to 10 carbon atoms or a group containing an oxygen atom; and

plural Rs, R¹s and R²s in the formula (I) may be the same or different:

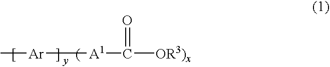

wherein Ar is an arylene group having 6 to 10 carbon atoms, a group formed by combining two or more arylene groups having 6 to 10 carbon atoms, or a group formed by combining one or more of an arylene group having 6 to 10 carbon atoms and at least one of an alkylene group and an ether group;

A¹ is a single bond, an alkylene group, an ether group or a group formed by combining two or more of an alkylene group and an ether group;

R³s are independently hydrogen, a substituted or unsubstituted straight-chain aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group, or a group having a structure in which these groups and a divalent group (a substituted or unsubstituted alkylene group, a substituted or unsubstituted arylene group, a substituted or unsubstituted silylene group, a group formed by bonding two or more of these groups or a group formed by combining one or more of these groups and one or more of an ester group, a carbonic ester group and an ether group) are bonded;

x is an integer of 1 to 5;

y is an integer of 0 to 3; and
plural R³s, Ars, A¹s, xs and ys may be the same or different, providing that a case where all of R¹ and R² are hydrogen and R is a 4-carboxyphenyl group or a 4-(carboxylmethyleneoxy)phenyl group is excluded.
   2. The cyclic compound according to 1, wherein the group shown by the formula (1) is any of groups shown by the following formulas (1-1) to (1-6):

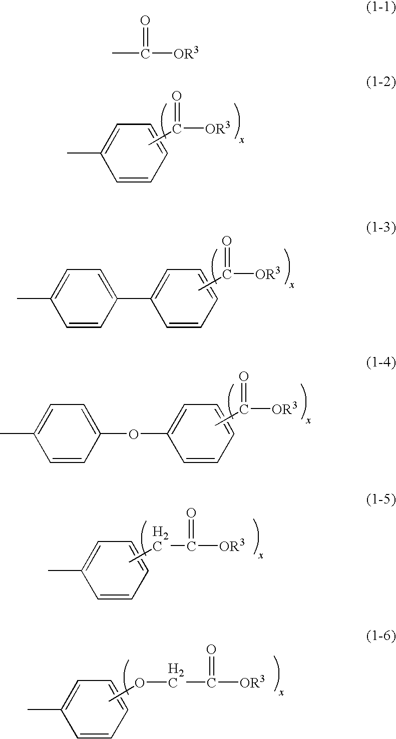

wherein R³s are independently hydrogen, a substituted or unsubstituted straight-chain aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group or a group having a structure in which these groups and a divalent group (a substituted or unsubstituted alkylene group, a substituted or unsubstituted arylene group, a substituted or unsubstituted silylene group, a group formed by bonding two or more of these groups or a group formed by combining one or more of these groups and one or more of an ester group, a carbonic ester group and an ether group) are bonded; and

x is an integer of 1 to 5.

3. The cyclic compound according to 1 or 2, wherein at least one of R¹s or at least one of R³s is an acid-dissociative dissolution inhibiting group, and the acid-dissociative dissolution inhibiting group is a group selected from an alkoxycarbonyl group, an alkoxycarbonylmethyl group, an alkoxymethyl group, an alkoxyalkylmethyl group, an alkoxyarylmethyl group, an alkoxycarbonylphenyl group, a bis(alkoxycarbony)phenyl group and a tris(alkoxycarbonyl)phenyl group.
4. The cyclic compound according to 3, wherein the acid-dissociative dissolution inhibiting group is a group selected from groups shown by the following formulas (2) to (17):

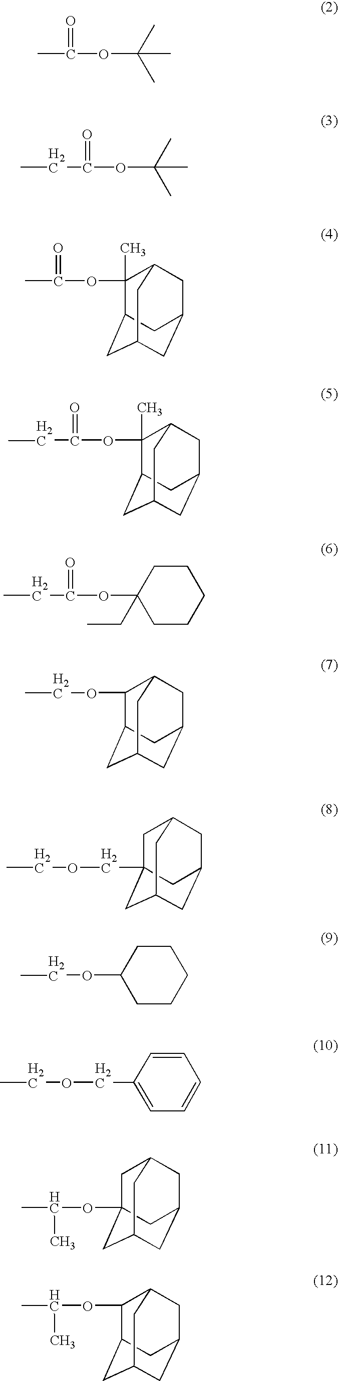

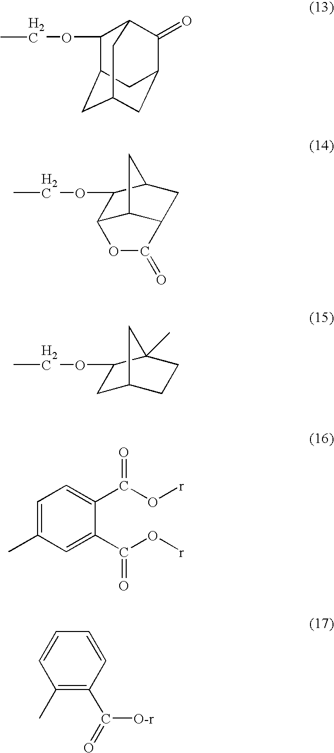

wherein r in the formulas (16) and (17) is a monovalent group selected from groups shown by the above formulas (2) to (15) and the following formulas (18) to (20):

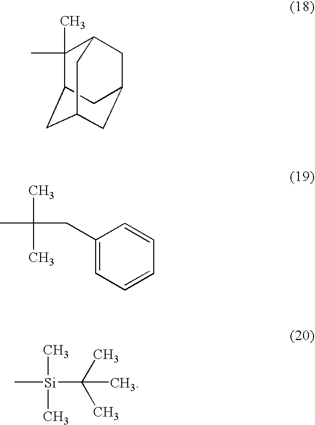

5. A photoresist base material comprising the cyclic compound according to any of 1 to 4.
6. A photoresist composition comprising the photoresist base material according to 5 and a solvent.
7. The photoresist composition according to 6 which further comprises a photo-acid generator.
8. The photoresist composition according to 6 or 7 which further comprises a basic organic compound as a quencher.
9. A fine processing method using the photoresist composition according to any of 6 to 8.
10. A semiconductor apparatus which is produced by the fine processing method according to 9.

According to the invention, it is possible to provide a photoresist base material improved in solubility in a solvent for coating and the composition thereof. If ultrafine processing by lithography using extreme ultraviolet light or an electron beam is conducted by using the photoresist base material, and the composition thereof of the invention, patterns can be formed with a high sensitivity, a high contrast and a low line edge roughness.

### BRIEF DESCRIPTION OF THE DRAWINGS

FIG. 1 is an electron microscopic photograph of a silicon wafer on which a pattern was formed in Evaluation 2;

FIG. 2 is a ¹H-NMR spectrum of a compound (XIV) synthesized in Example 10;

FIG. 3 is a ¹H-NMR spectrum of a compound (XV) synthesized in Example 11;

FIG. 4 is a ¹H-NMR spectrum of a compound (XVI) synthesized in Example 12;

FIG. 5 is a ¹H-NMR spectrum of a compound (XVII) synthesized in Example 13; and

FIG. 6 is a ¹H-NMR spectrum of a compound (XVIII) synthesized in Example 14.

### BEST MODE FOR CARRYING OUT THE INVENTION

The cyclic compound of the invention has a structure shown by the following formula (I):

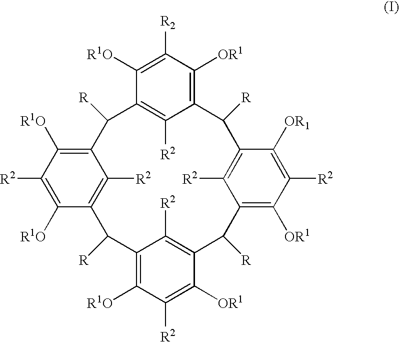

In the formula (I), R is a group shown by the following formula (1):

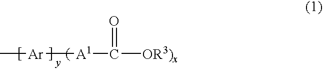

In the formula (1), Ar is a substituted or unsubstituted arylene group having 6 to 10 carbon atoms, a group formed by combining two or more arylene groups having 6 to 10 carbon atoms or a group formed by combining one or more of an arylene group having 6 to 10 carbon atoms and at least one of an alkylene group and an ether group. Preferred examples include phenylene, methylphenylene, dimethylphenylene, trimethylphenylene, tetramethylphenylene, naphthylene, biphenylene and oxydiphenylene.

Of these, phenylene, biphenylene and oxydiphenylene are preferable.

A¹ is a single bond, an alkylene group, an ether group or a group formed by combining two or more of an alkylene group and an ether group.

As the alkylene group, those having 1 to 4 carbon atoms such as a methylene group, a dimethylmethylene group, an ethylene group, a propylene group and a butylene group are preferable.

Preferred examples of the group formed by combining two or more of an alkylene group and an ether group include an oxymethylene group, an oxydimethylmethylene group, an oxyethylene group, an oxypropylene group and an oxybutylene group.

It is preferred that A¹ be a single bond or an oxymethylene group (—O—CH₂—).

R³s are independently hydrogen, a straight-chain aliphatic hydrocarbon group having 1 to 20 carbon atoms, a branched aliphatic hydrocarbon group having 3 to 12 carbon atoms, a cyclic aliphatic hydrocarbon group having 3 to 20 carbon atoms, an aromatic group having 6 to 10 carbon atoms, an alkoxyalkyl group, a silyl group, or a group having a structure in which these groups and a divalent group are bonded.

Preferred examples of the straight-chain aliphatic hydrocarbon group having 1 to 20 carbon atoms include a methyl group, an ethyl group, a propyl group, a butyl group, a pentyl group, a hexyl group, a heptyl group and an octyl group.

Preferred examples of the branched aliphatic hydrocarbon group having 3 to 12 carbon atoms include a t-butyl group, an iso-propyl group, an iso-butyl group and a 2-ethylhexyl group.

Preferred examples of the cyclic aliphatic hydrocarbon group having 3 to 20 carbon atoms include a cyclohexyl group, a norbonyl group, an adamantyl group, a biadamantyl group and a diadamantyl group.

Preferred examples of the aromatic group having 6 to 10 carbon atoms include a phenyl group and a naphthyl group.

Preferred examples of the alkoxyalkyl group include a methoxymethyl group, an ethoxymethyl group and an adamantyloxymethyl group.

Preferred examples of the silyl group include a trimethylsilyl group and a t-butyldimethylsilyl group.

Each of the above groups may have a substituent. Specific examples thereof include an alkyl group such as a methyl group and an ethyl group, a ketone group, an ester group, an alkoxy group, a nitrile group, a nitro group and a hydroxyl group.

R³ may be a group having a structure in which each of the above-mentioned groups and a divalent group are bonded.

Examples of the divalent group include a substituted or unsubstituted alkylene group, a substituted or unsubstituted arylene group, a substituted or unsubstituted silylene group, a group formed by bonding two or more of these groups, and a group formed by combining one or more of these groups and one or more of an ester group, a carbonic ester group and an ether group.

Preferred examples of the alkylene group include a methylene group and a methylmethylene group, and preferred examples of the arylene group include a phenylene group.

As the divalent group, a group having the following structure is preferable.

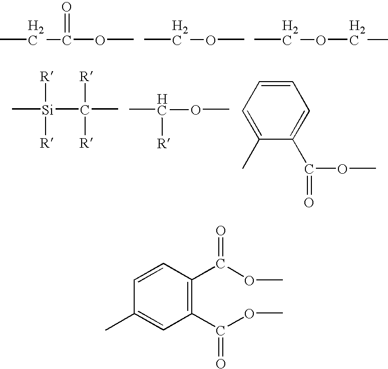

wherein R′ s independently represent H or an alkyl group.

x is an integer of 1 to 5, with an integer of 1 to 3 being preferable.

y is an integer of 0 to 3, with an integer of 1 or 2 being preferable.

In the formula (I), plural Rs are present. Each of R³s, Ars, A¹s, xs and ys which constitute R may be the same or different.

In the invention, it is preferred that the group shown by the formula (1) be any of groups shown by the following formulas (1-1) to (1-6):

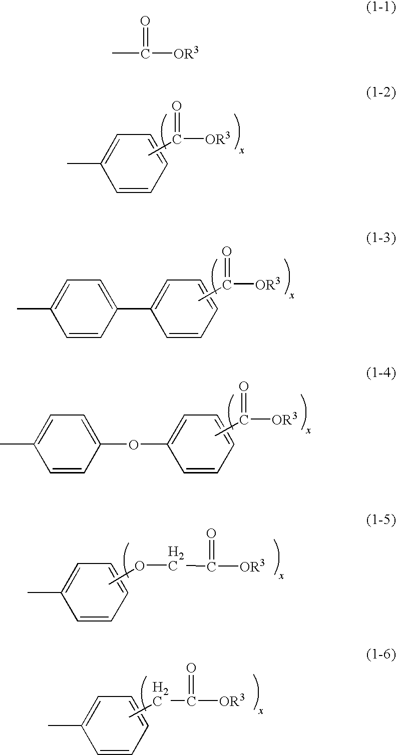

wherein R³ represents the same group as that in the formula (1) and x is an integer of 1 to 5.

R¹s are independently hydrogen, a straight-chain aliphatic hydrocarbon group having 1 to 20 carbon atoms, a branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group or a group having a structure in which these groups and a divalent group (a substituted or unsubstituted alkylene group, a substituted or unsubstituted arylene group, a substituted or unsubstituted silylene group, a group formed by bonding two or more of these groups or a group formed by combining one or more of these groups and one or more of an ester group, a carbonic ester group and an ether group) are bonded.

Preferred examples of each group of R¹ are the same as those for R³ mentioned above.

R²s are independently hydrogen, a group shown by —O—R¹, a straight-chain aliphatic hydrocarbon group having 1 to 20 carbon atoms, a branched aliphatic hydrocarbon group having 3 to 12 carbon atoms, a cyclic aliphatic hydrocarbon group having 3 to 20 carbon atoms, an aromatic group having 6 to 10 carbon atoms or a group containing an oxygen atom.

Preferred examples of the straight-chain aliphatic hydrocarbon group having 1 to 20 carbon atoms, the branched aliphatic hydrocarbon group having 3 to 12 carbon atoms, the cyclic aliphatic hydrocarbon group having 3 to 20 carbon atoms and the aromatic group having 6 to 10 carbon atoms are the same as those for R³ mentioned above.

As the group containing an oxygen atom, a group shown by —O—R¹, an alkoxy group, an alkoxycarbonyl group or the like are preferable.

Plural Rs, R¹s and R²s in the formula (I) may be the same or different.

In the above-mentioned formula (I), a compound wherein all of R¹ and R² are hydrogen and R is a 4-carboxyphenyl group or a 4-(carboxymethyleneoxy)phenyl group is outside the scope of the invention.

The cyclic compound shown by the above-mentioned formula (I) is useful as a photoresist base material, in particular, as a photoresist base material used in ultrafine processing by lithography by extreme ultraviolet light (wavelength: 15 nm or less) or by an electron beam.

In the cyclic compound of the invention which has both a carboxylic acid structure and a phenol structure, by selecting a central skeleton structure, the position at which a protective group is introduced or the number of a protective group to be introduced can be easily controlled. As a result, a base material composed of a controlled single structure can be obtained easily.

As a result, in respect of resolution, the photoresist base material of the invention contributes to low line edge roughness due to high dissolution controllability in a developer. Further, the cyclic compound of the invention has improved adhesiveness to a substrate and a high thin film strength.

The photoresist base material of the invention may be used singly or in combination of two or more within a range which does not impair the advantageous effects of the invention.

In the compound of the invention, it is preferred that at least one of R¹s or at least one of R³s be an acid-dissociative dissolution inhibiting group. Due to a high degree of reactivity for EUVL and an electron beam, the acid-dissociative dissolution inhibiting group is improved in sensitivity, as well as in etching resistance. Therefore, it can be preferably used as a photoresist base material for ultrafine processing.

Examples of the acid-dissociative dissolution inhibiting group include an alkoxycarbonyl group, an alkoxycarbonylmethyl group, an alkoxymethyl group, an alkoxyalkylmethyl group, an alkoxyarylmethyl group, an alkoxycarbonylphenyl group, a bis(alkoxycarbonyl)phenyl group or a tris(alkoxycarbonyl)phenyl group, a silyl group or a group having a structure in which these groups and the divalent group are bonded.

In particular, it is preferred that the acid-dissociative dissolution inhibiting group be a group selected from groups shown by the following formulas (2) to (17):

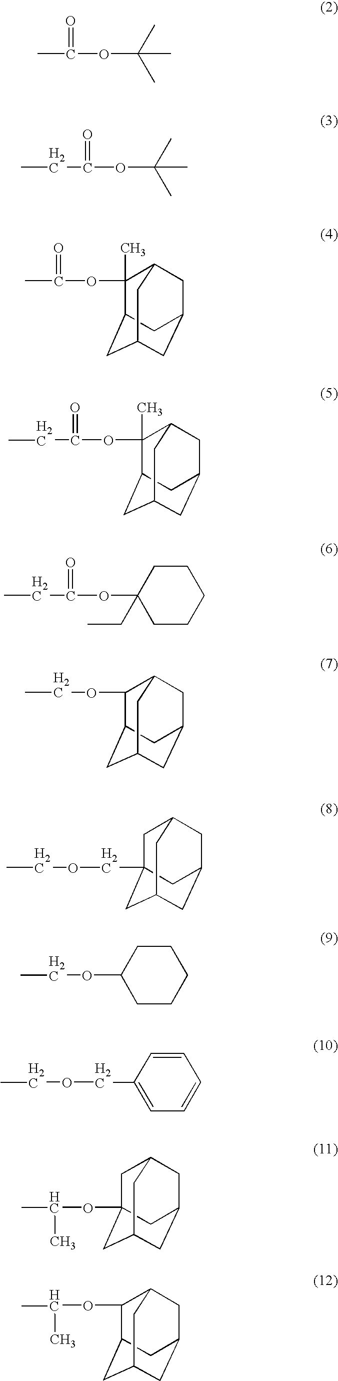

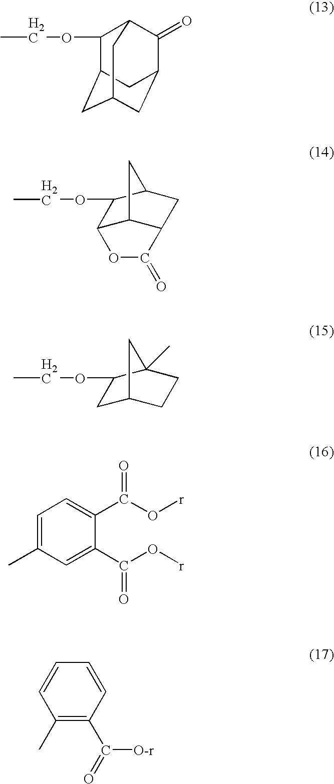

(r in the formulas (16) and (17) is a monovalent group selected from groups shown by the above-mentioned formulas (2) to (15) and the following formulas (18) to (20))

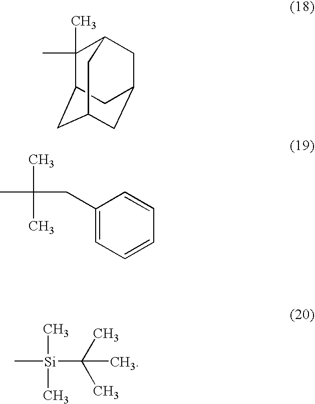

The photoresist base material containing the cyclic compound which has the above-mentioned acid-dissociative dissolution inhibiting group is capable of reducing the amount of a resist outgas, in particular. The reason therefor is that, since the acid-dissociative dissolution inhibiting group has a relatively large molecule having a molecular weight of not less than 100 and not more than 1000 and has a cyclic main structure, a low-molecular compound constituting a resist outgas is hard to be released.

The cyclic compound of the invention can be synthesized by a known method, in which, for example, a calixresorcinarene derivative (precursor) is synthesized by subjecting an aldehyde compound having corresponding structures and an aromatic compound containing a hydroxyl group to a condensation/annulation reaction in the presence of an acid catalyst, and a compound corresponding to a group such as R is introduced to the precursor by an esterification reaction, an etherification reaction, an acetalification reaction or the like. The specific examples of the synthesis method will be explained in Examples given later.

The compounds having a structure corresponding to the formulas (2) to (17) are known compounds, or compounds capable of being synthesized by a known production method.

The cyclic compound of the invention becomes amorphous under conditions where the compound is used as a photoresist base material (normally, at room temperature). Therefore, if used as a base material, the cyclic compound of the invention is preferable in respect of applicability as a photoresist composition or strength of a photoresist film.

When used in processing of 20 to 50 nm, which is characteristic ultrafine processing by extreme ultraviolet light or an electron beam, the base material of the invention can suppress line edge roughness to 2 nm or less, preferably 1 nm or less (3 σ). The reason therefor is that, the average diameter of the molecule of the cyclic compound of the invention is smaller than the value of line edge roughness (5 nm or less) required for the size of a desired pattern, specifically, 100 nm or less, in particular, 50 nm or less.

When the cyclic compound is used as a photoresist base material, it is preferred that the cyclic compound be purified to remove basic impurities or the like (for example, alkali metal ions such as ammonia, Li, Na and K, alkaline earth metal ions such as Ca and Ba). At this time, it is preferred that the amount of impurities be reduced to 1/10 or less of the amount of impurities contained before the purification. Specifically, the amount of the basic impurities is preferably 10 ppm or less, more preferably 2 ppm or less. By reducing the amount of basic impurities to 10 ppm or less, the sensitivity to extreme ultraviolet light or an electron beam of the photoresist base material composed of this compound is significantly increased, whereby a fine processing pattern of the photoresist composition can be preferably formed by lithography.

As the method for purification, cleaning with an aqueous acidic solution, re-precipitation using an ion-exchange resin or ultrapure water can be given. Purification may be conducted by combination of these methods. For example, after cleaning using an aqueous acetic acid solution as an aqueous acidic solution, a re-precipitation treatment is conducted using an ion exchange resin or ultrapure water.

As for the type of the aqueous acidic solution or the ion exchange resin used, an optimum one may be appropriately selected according to the amount or type of basic impurities to be removed or the type of a basic material to be treated.

The photoresist composition of the invention contains the above-mentioned photoresist base material of the invention and a solvent for dissolving the photoresist base material to form a liquid composition. In order to apply uniformly by a technique such as spin coating, dip coating and painting to a substrate or the like to which ultrafine processing is conducted, the photoresist composition is required to be in the form of a liquid composition.

As the solvent, those which are commonly used in the field of a photoresist can be used. Preferred examples include glycols such as 2-methoxyethyl eter, ethylene glycol monomethyl ether, propylene glycol monomethyl ether and propylene glycol methyl ether acetate; lactic acid esters such as ethyl lactate and methyl lactate; propionates such as methyl propionate and ethyl propionate; cellosolve esters such as methyl cellosolve acetates; aromatic hydrocarbons such as toluene and xylene, ketones such as methyl amyl ketone, methyl ethyl ketone, cyclohexanone and 2-heptanone; butyl acetate, or a mixed solvent of two or more of these.

The solvent to be used can be appropriately selected according to the solubility, film-forming properties or the like of the photoresist base material.

The photoresist composition of the invention does not particularly require an additive if the molecule of the base material contains a chromophore which is active to EUVL and/or electron beams and thus exhibits properties as a photoresist by itself. However, if the performance (sensitivity) as a photoresist is required to be increased, a photoacid generator (PAG) or the like may be added as a chromophere.

As the photoacid generator, in addition to known photoacid generators shown by the following structures, other compounds which have the similar activity may be commonly used. The type and amount of the preferable PAG can be specified according to the base material of the invention, the shape, size or the like of a desired fine pattern.

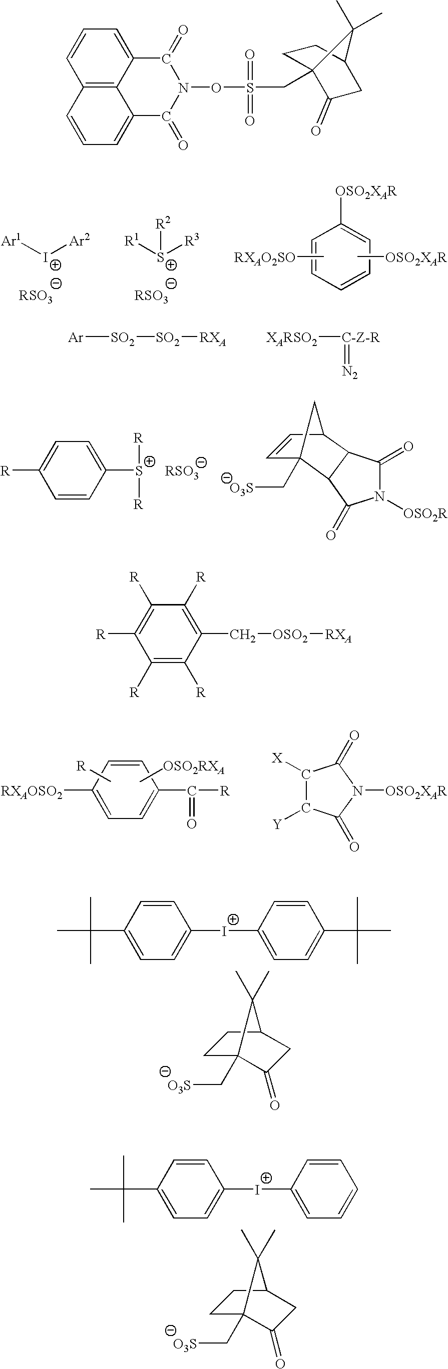

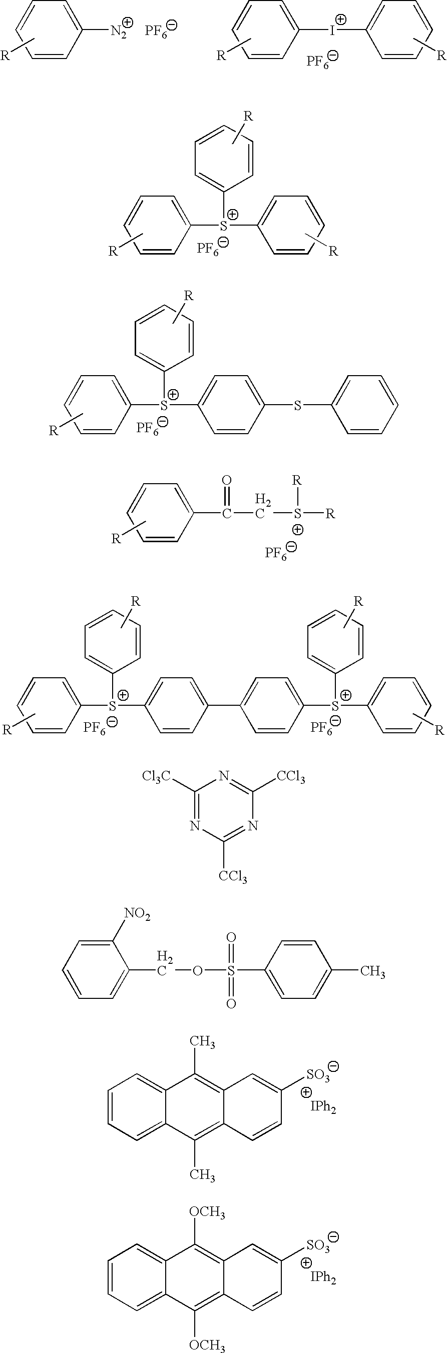

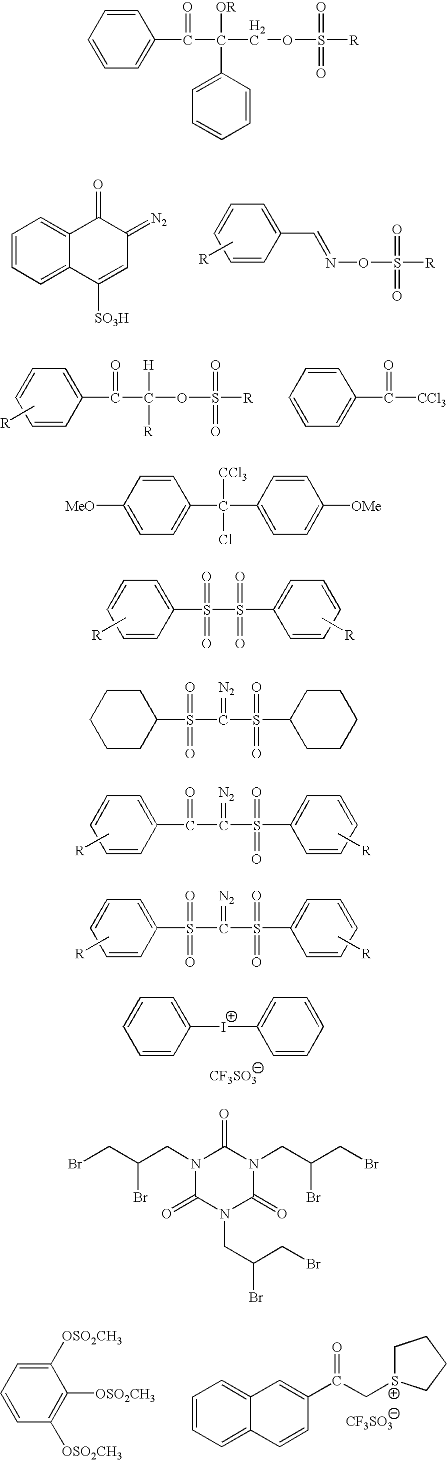

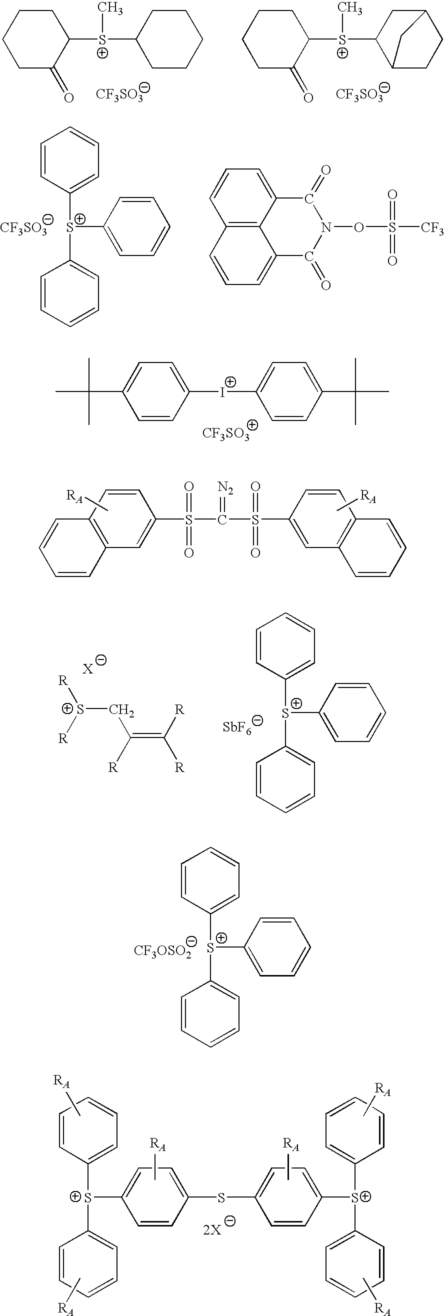

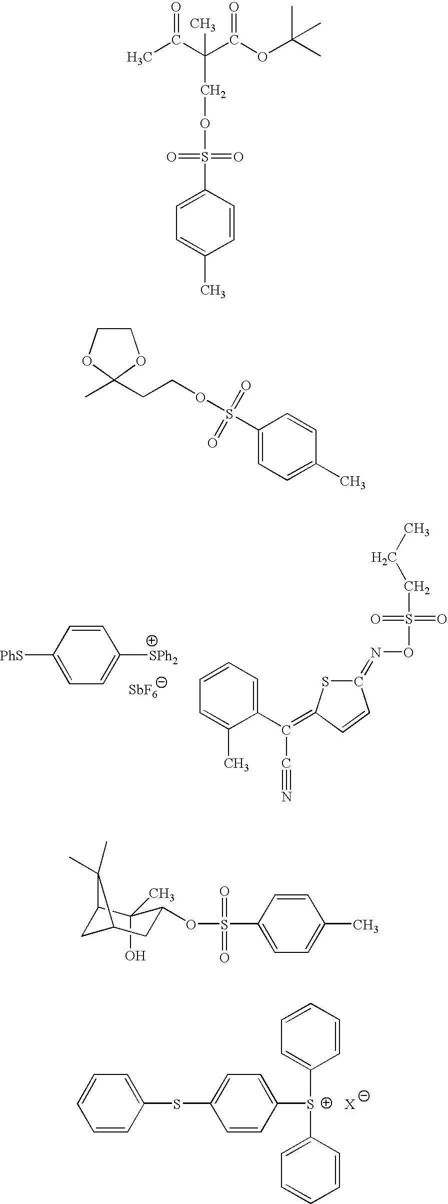

wherein Ar, Ar¹ and Ar² are independently a substituted or unsubstituted aromatic group having 6 to 20 carbon atoms; R, R¹, R², R³ and R_{A} are independently a substituted or unsubstituted aromatic group having 6 to 20 carbon atoms, a substituted or unsubstituted aliphatic group having 1 to 20 carbon atoms, and X, X_{A}, Y and Z are independently an aliphatic sulfonium group, an aliphatic sulfonium group containing fluorine, a tetrafluoroborate group and a hexafluorophosphonium group.

Generally, a PAG is used in an amount range of 0.1 to 20 wt % relative to the photoresist base material.

If need arises, a quencher may be added which suppresses an over reaction of a PAG. As a result, sensitivity to extreme ultraviolet light or resolution for an electron beam can be improved. As the quencher, in addition to known quenchers, other compounds which have a similar activity may be generally used.

As the quencher, in respect of solubility in a photoresist composition or dispersibility or stability in a photoresist layer, it is preferable to use a basic organic compound. Specific examples of the basic organic compound include pyridines such as quinoline, indole, pyridine and bipyridine, pyrimidines, pyrazines, piperidine, piperazine, pyrrolidine, 1,4-diazabicyclo[2.2.2]octane, aliphatic amines such as triethylamine and trioctylamine, and tetrabutylammonium hydroxide.

The type and amount of the preferable quencher can be specified according to the base material of the invention, PAG, the shape, size or the like of desired fine patterns.

A quencher is generally used in an amount of 10 to 1×10⁻³ wt % relative to the photoresist base material, or in an amount of 50 to 0.01 wt % relative to the PAG.

A photosensitive aid, a plasticizer, a speed promoter, a photosensitizer, a sensitizer, an acid growth function material, an etching resistance reinforcing agent or the like may be added to the photoresist composition of the invention. These additives may be used as a mixture of a plurality of components having the same function, a mixture of a plurality of component having different functions, or a mixture of precursors thereof. Although the content ratio of these additives cannot be specified unconditionally since it depends on the type of the components used, generally, these additives are used in a content ratio similar to that in known photoresists.

As for the amount of components other than the solvent in the composition, i.e. the amount of photoresist solid matters, it is preferred that the amount be one which is suitable for forming a photoresist layer in a desired thickness. Specifically, although the amount is generally 0.1 to 50 wt % of the total weight of the photoresist composition, it can be specified according to the type of the base material or the solvent used, or according to the desired thickness or the like of a photoresist layer.

One example of a fine processing method using the photoresist composition of the invention will be explained below. The photoresist composition of the invention is applied to a substrate as a liquid coating composition by a method such as spin coating, dip coating and painting. After the application, in order to remove the solvent, it is common that the coated material is dried by being heated to 80 to 160° C., for example, until the photoresist coating layer becomes non-sticky. Further, in order to improve adhesion with a substrate, hexamethyldisilazane (HMDS) or the like can be used as an intermediate layer. These conditions can be appropriately specified according to the type of a base material or a solvent used, the desired thickness of a photoresist layer, or the like.

After heating and drying, the substrate with the above-mentioned photoresist coating layer, which is no longer sticky, is exposed through a photomask by EUVL or is irradiated with an electron beam by an arbitral method to remove protective groups contained in the base material, thereby causing solubility differences between the exposed areas and unexposed areas in the photoresist coating layer. After the exposure, the substrate is baked to increase the solubility differences, followed by development with an alkaline developer or the like to form relief images. By these operations, ultrafine processing patterns are formed on the substrate. The above conditions can be determined according to the type of the base material or a solvent used, the desired thickness of a photoresist layer or the like.

If ultrafine processing is conducted using the photoresist composition of the invention by lithography with extreme ultraviolet light or an electron beam, patterns with isolated lines of 100 nm or less, particularly 50 nm or less, a 1:1 line-and-space (L/S), holes, or the like can be formed with a high degree of sensitivity, high contrast and low line edge roughness.

By the fine processing method of the invention, a semiconductor apparatus such as an ULSI, a large-capacity memory device and an ultra-high speed logic device can be produced.

### EXAMPLES

### Production Example 1

The precursor (1) of the cyclic compound shown by the following formula was synthesized:

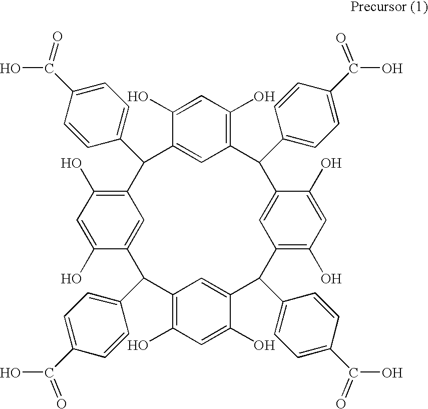

A four-neck flask with a capacity of 300 ml equipped with a nitrogen-introducing tube, a thermometer, a mechanical stirrer and a Dimroth condenser was charged with 5.51 g of resorcinol (50 mmol: manufactured by Wako Pure Chemical Industries, Ltd.) and 7.51 g of p-formylbenzoate (50 mmol: manufactured by Wako Pure Chemical Industries, Ltd.). Then, 40 ml of ethanol was added, followed by stirring. Nitrogen was introduced to allow the inside of the flask to be a nitrogen atmosphere. Subsequently, 10 ml of concentrated hydrochloric acid was added from a dripping funnel slowly such that the temperature inside the flask did not exceed 35° C. After the completion of the dropwise addition of the concentrated hydrochloric acid, the flask was heated to 80° C. (inside the flask) by immersing the flask in an oil bath, thereby to allow the mixture to react for 3 hours. Then, the heating was stopped, and the inside of the reaction flask was cooled to around room temperature. A solid which was generated by the reaction was filtered and washed with a small amount of ethanol.

The resulting solid was transferred to a beaker with a capacity of 200 to 300 ml. Then, 100 ml of de-ionized water was added, followed by stirring by means of a magnetic stirrer for about 10 minutes. After the completion of the stirring, the solid was filtered again, and washed with de-ionized water. The same operation was repeated once again. After confirming that the filtrate was neutral, the solid was dried under vacuum for 16 hours. 6.41 g of the resulting white crystals were placed in a round-bottom flask with a capacity of 300 ml. Then, 80 ml of N,N-dimethylformamide (DMF) was added, followed by heating in an oil bath of 65° C. while stirring by means of a magnetic stirrer to allow the crystals to be dissolved completely.

After allowing the resultant to stand overnight, acetone was gradually added while stirring until the turbidity disappeared. Then, the mixture was allowed to stand for one day. The resulting crystals were taken out by filtration, washed with a small amount of acetone, and collected by drying under vacuum for 16 hours.

As a result of ¹H-NMR, it was confirmed that the collected compound was the above-mentioned precursor (1) (calixresorcinarene derivative) (amount: 1.73 g (1.79 mmol), yield: 14%).

The spectrum data of ¹H-NMR is given below.

¹H-NMR (tetramethylsilane as an internal standard: solvent (CD₃)₂SO: ppm): 5.49 (2H, s), 5.58 (4H, s), 6.14 (2H, s), 6.34 (2H, s), 6.40 (2H, s), 6.70 (8H, d), 7.47 (8H, d), 8.58 (4H, s), 8.77 (4H, s), 12.26 (4H, bs)

### Production Example 2

The precursor (2) of the cyclic compound shown by the following formula was synthesized.

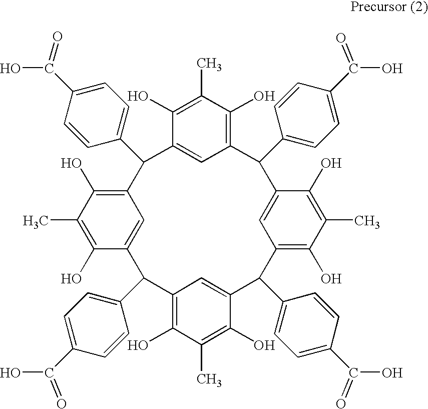

A four-neck flask with a capacity of 500 ml equipped with a nitrogen-introducing tube, a thermometer, a mechanical stirrer and a Dimroth condenser was charged with 24.8 g of 2-methyl resorcinol (0.2 mol: manufactured by Tokyo Chemical Industry Co., Ltd.) and 30.0 g of p-formylbenzoate (0.2 mol: manufactured by Wako Pure Chemical Industries, Ltd.). Then, 160 ml of ethanol was added, followed by stirring. Nitrogen was introduced to allow the inside of the flask to be a nitrogen atmosphere. The flask was cooled by immersing it in an ice/water bath until the temperature inside the flask became 5° C. Then, 40 ml of concentrated hydrochloric acid was added slowly from a dripping funnel such that the temperature inside the flask did not exceed 20° C. After the completion of the dropwise addition of the concentrated hydrochloric acid, cooling was stopped, and the flask was heated to 80° C. (inside the flask) by immersing the flask in an oil bath, thereby to allow the mixture to react for 3 hours. Then, the heating was stopped, and the inside of the reaction flask was cooled to around room temperature. A solid which was generated by the reaction was filtered and washed with a small amount of ethanol. The resulting solid was transferred to a beaker with a capacity of 1 l. Then, 300 ml of de-ionized water was added, followed by stirring with a magnetic stirrer for about 10 minutes. After the completion of the stirring, the solid was filtered again, and washed with de-ionized water. The same operation was repeated once again. After confirming that the filtrate was neutral, the solid was dried under vacuum for 16 hours.

25.1 g of the resulting beige crystals were placed in a round-bottom flask with a capacity of 1 l. Then, 500 ml of DMF was added, followed by heating in an oil bath of 85° C. with stirring by means of a magnetic stirrer to allow the crystals to be dissolved. Then, the resultant was allowed to stand for one day. The generated crystals were taken out by filtration, washed with a small amount of DMF, and dried under vacuum for 16 hours. As a result of ¹H-NMR, it was confirmed that the collected compound was the above-mentioned precursor (2) (amount: 10.6 g (10.3 mmol), yield: 21%).

The spectrum data of ¹H-NMR is given below.

¹H-NMR (tetramethylsilane as an internal standard: solvent (CD₃)₂SO: ppm): 1.895 (6H, s), 2.13 (6H, s), 5.36 (2H, s), 5.71 (4H, s), 6.21 (2H, s), 6.75 (8H, d), 7.47 (8H, d), 7.58 (4H, bs), 7.66 (4H, bs), 12.27 (4H, bs)

### Production Example 3

The precursor (3) of the cyclic compound shown by the following formula was synthesized.

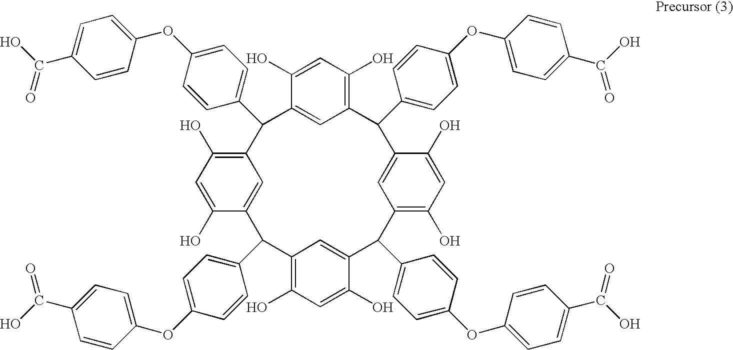

### Step 1: Synthesis of an Ethyl Ester of the Precursor (3)

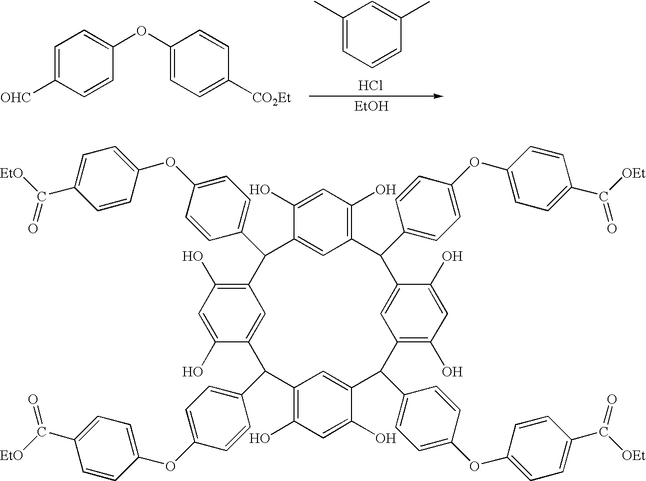

A four-neck flask with a capacity of 300 ml equipped with a nitrogen-introducing tube, a thermometer, a mechanical stirrer and a Dimroth condenser was charged with 6.61 g of resorcinol (60 mmol: manufactured by Wako Pure Chemical Industries, Ltd.) and 16.22 g of 4-(4′-formylphenyloxy)ethyl benzoate (60 mmol: synthesized by a method described in SYNTHESIS, 1, 1991, pp. 63-68). Then, 48 ml of ethanol was added, followed by stirring. Nitrogen was introduced to allow the inside of the flask to be a nitrogen atmosphere. Subsequently, 12 ml of concentrated hydrochloric acid was added from a dripping funnel slowly such that the temperature inside the flask did not exceed 35° C. After the completion of the dropwise addition of the concentrated hydrochloric acid, the flask was heated to 80° C. (inside the flask) by immersing the flask in an oil bath, thereby to allow the mixture to react for 6 hours.

Then, the heating was stopped, and the inside of the reaction flask was cooled to around room temperature. A solid which was generated by the reaction was filtered and washed with a small amount of ethanol. The resulting solid was transferred to a beaker with a capacity of 300 ml. Then, 100 ml of de-ionized water was added, followed by stirring by means of a magnetic stirrer for about 10 minutes. After the completion of the stirring, the solid was filtered again, and washed with de-ionized water. The same operation was repeated once again. After confirming that the filtrate was neutral, the solid was dried under vacuum for 16 hours. An ethyl ester of the precursor (3) was obtained as a white solid in an amount of 20.89 g.

### Step 2: Hydrolysis

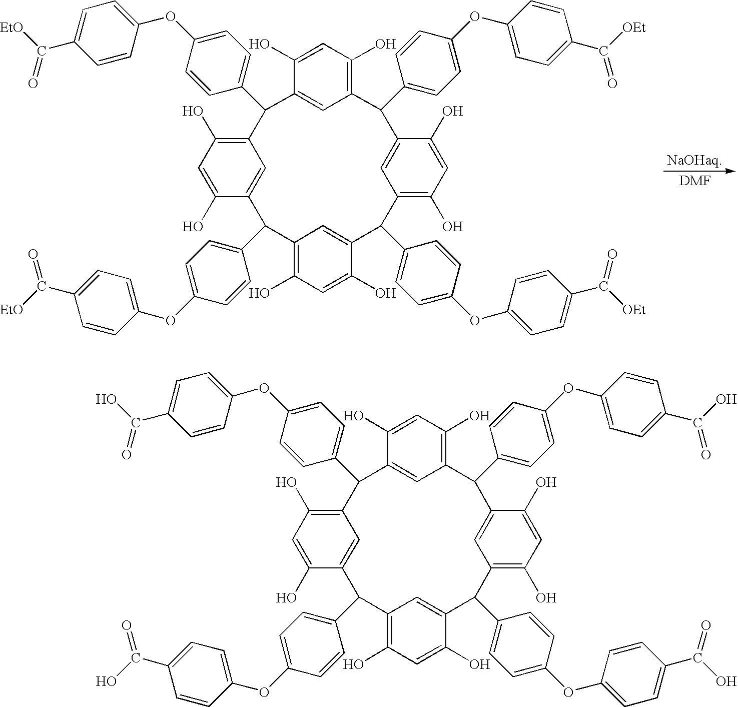

20.89 g of the ethyl ester of the precursor (3) obtained in the above-mentioned step (1) was placed in a round-bottom flask with a capacity of 1 l. Then, 300 ml of DMF was added, followed by stirring. Subsequently, 15 g of sodium hydroxide which had been dissolved in 75 ml of de-ionized water was added to the flask. The mixture was heated in an oil bath of 65° C. while stirring by means of a magnetic stirrer, thereby to allow the mixture to react for 6 hours. After cooling the flask to around room temperature, the contents of the flask were diluted by pouring them to 1.2 l of de-ionized water in a beaker with a capacity of 2 l. Then, an aqueous 10% hydrochloric acid solution was added to allow the pH to be 1. A solid which was generated was taken out by filtration, washed with de-ionized water and dried under vacuum, whereby 19.6 g of a white solid was obtained.

The resulting white solid was placed in a round-bottom flask with a capacity of 300 ml. Then, 80 ml of DMF was added, followed by heating in an oil bath of 65° C. while stirring by means of a magnetic stirrer to allow the solid to be dissolved. After allowing the resultant to stand overnight, de-ionized water was gradually added while stirring until the turbidity disappeared. Then, the mixture was allowed to stand for one day. The resulting crystals were taken out by filtration, washed with a small amount of a mixed solvent of DMF and de-ionized water, and dried under vacuum for 16 hours. As a result of ¹H-NMR, it was confirmed that the resulting compound was the above-mentioned precursor (3) (amount: 11.46 g (8.57 mmol), yield: 57%).

The spectrum data of ¹H-NMR is given below.

¹H-NMR (tetramethylsilane as an internal standard: solvent (CD₃)₂SO: ppm): 5.59 (4H, s), 5.77 (2H, s), 6.18 (2H, s), 6.33 (2H, s), 6.39 (2H, s), 6.75 (16H, dd), 6.84 (8H, d), 7.89 (8H, d), 8.58 (4H, bs), 8.65 (4H, bs), 12.72 (4H, bs)

### Production Example 4

The precursor (4) of the cyclic compound shown by the following formula was synthesized.

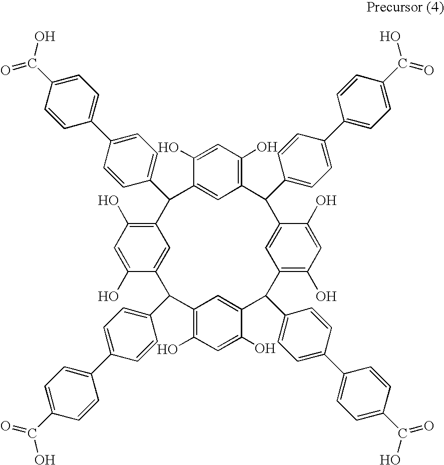

### Step 1: Synthesis of an Ethyl Ester of the Precursor (4)

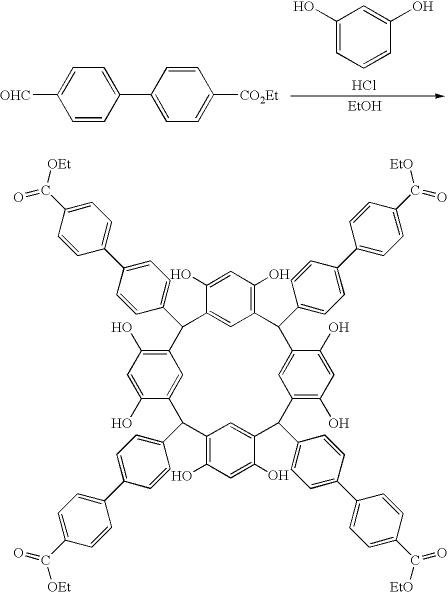

A four-neck flask with a capacity of 300 ml equipped with a nitrogen-introducing tube, a thermometer, a mechanical stirrer and a Dimroth condenser was charged with 7.36 g of resorcinol (67 mmol: manufactured by Wako Pure Chemical Industries, Ltd.) and 17.0 g of 4-(4′-formylphenyl)ethyl benzoate (67 mmol: synthesized by a method described in Bioorganic & Medicinal Chemistry Letters, 13, 16, 2003, pp. 2651-2654). Then, 55 ml of ethanol was added, followed by stirring. Nitrogen was introduced to allow the inside of the flask to be a nitrogen atmosphere. Subsequently, 13.5 ml of concentrated hydrochloric acid was added from a dripping funnel slowly such that the temperature inside the flask did not exceed 35° C. After the completion of the dropwise addition of the concentrated hydrochloric acid, the flask was heated to 80° C. (inside the flask) by immersing the flask in an oil bath, thereby to allow the mixture to react for 6 hours.

Then, the heating was stopped, and the inside of the reaction flask was cooled to around room temperature. A solid which was generated by the reaction was filtered and washed with a small amount of ethanol. The resulting solid was transferred to a beaker with a capacity of 300 ml. Then, 100 ml of de-ionized water was added, followed by stirring with a magnetic stirrer for about 10 minutes. After the completion of the stirring, the solid was filtered again, and washed with de-ionized water. The same operation was repeated once again. After confirming that the filtrate was neutral, the solid was dried under vacuum for 16 hours. An ethyl ester of the precursor (4) was obtained as a white solid in an amount of 22.21 g.

### Step 2: Hydrolysis

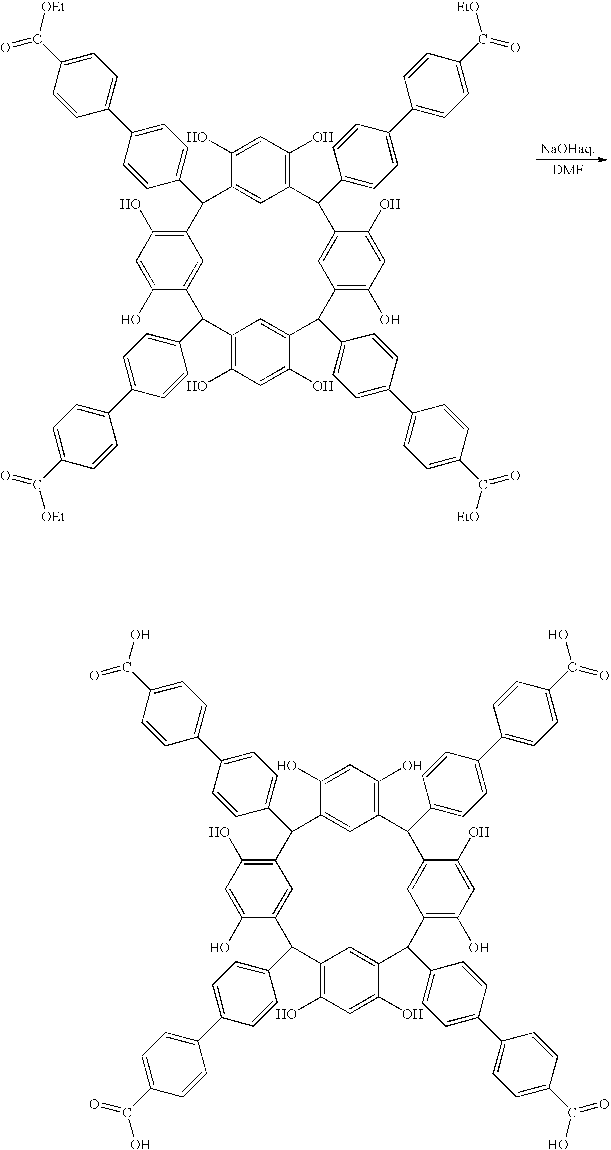

22.21 g of the ethyl ester of the precursor (4) obtained in the above-mentioned step (1) was placed in a round-bottom flask with a capacity of 1 l. Then, 320 ml of DMF was added. 13 g of sodium hydroxide which had been dissolved in 65 ml of de-ionized water was added to the flask while stirring by means of a magnetic stirrer. The mixture was heated in an oil bath of 65° C., thereby to allow the mixture to react for 3 hours. After cooling the flask to around room temperature, the contents of the flask were diluted by pouring them into 1.2 l of de-ionized water in a beaker with a capacity of 2 l. Then, an aqueous 10% hydrochloric acid solution was added to allow the pH to be 1. A solid which was generated was taken out by filtration, washed with de-ionized water and dried under vacuum, whereby 20.2 g of a white solid was obtained.

20.2 g of the resulting white solid was placed in a round-bottom flask with a capacity of 2 l. Then, 630 ml of DMF was added, followed by heating in an oil bath of 85° C. while stirring by means of a magnetic stirrer to allow the crystals to be dissolved. After allowing the resultant to stand overnight, the generated crystals were taken out by filtration, washed with a small amount of DMF, and dried under vacuum for 16 hours. As a result of the ¹H-NMR, it was confirmed that the resulting compound was the above-mentioned precursor (4) (amount: 5.88 g (4.62 mmol), yield: 28%).

The spectrum data of ¹H-NMR is given below.

¹H-NMR (tetramethylsilane as an internal standard: solvent (CD₃)₂SO: ppm): 5.63 (4H, s), 5.81 (2H, s), 6.17 (2H, s), 6.44 (2H, s), 6.46 (2H, s), 6.77 (8H, d), 7.21 (8H, d), 7.34 (8H, d), 7.70 (8H, d), 8.56 (4H, bs), 8.72 (4H, bs), 12.69 (4H, bs)

### Example 1

The cyclic compound shown by the following formula (IV) was synthesized.

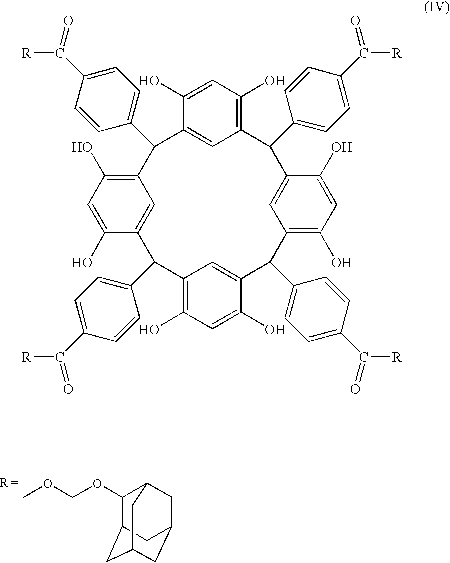

A three-neck flask (capacity: 200 ml) equipped with a thermometer, which had been replaced with a nitrogen gas, was charged with 0.71 g (0.73 mmol) of the precursor (1) synthesized in the above-mentioned Production Example 1. Then, 7 ml of DMF and 0.37 g (3.66 mmol) of triethylamine were added, followed by stirring. The flask was then cooled in an ice/water bath to allow the temperature inside of the flask to be 4° C. Then, 0.735 g (3.66 mmol: synthesized by a method described in SYNTHESIS, 11, 1982, pp. 942-944) of 2-chloromethoxyadamantane was dissolved in 7 ml of DMF. The resultant was added dropwise to the flask slowly such that the temperature did not exceed 10° C. After the completion of the dropwise addition, the cooling was stopped. When the inside temperature became around room temperature, the mixture was allowed to react in a nitrogen atmosphere for 16 hours. About 100 ml of ice water was poured to the reaction solution, and the resultant was further stirred for one hour. A yellowish white solid which was generated was taken out by filtration, washed with de-ionized water, and dried under vacuum. The resulting 1.09 g of the yellowish white solid was purified by column chromatography (Merk Silica Gel 60; developing solvent; methylene chloride:methanol=10:1), whereby 0.576 g of a white crystal was obtained. As a result of ¹H-NMR, it was confirmed that the resulting crystal was the cyclic compound shown by the above-formula (IV) in which R is an adamantyl-2-yl-oxymethyleneoxy group (amount: 0.58 g (0.36 mmol), yield: 49%).

The spectrum data of ¹H-NMR is given below.

¹H-NMR (tetramethylsilane as an internal standard: solvent (CD₃)₂SO: ppm): 1.43-1.46 (8H, m), 1.66-1.68 (16H, m), 1.74-1.81 (16H, m), 1.95-2.00 (16H, m), 3.84 (4H, s), 5.14 (2H, s), 5.52-5.57 (12H, m), 6.16 (2H, s), 6.27 (2H, s), 6.40 (2H, s), 6.70 (8H, d), 7.45 (8H, d), 8.63 (4H, s), 8.79 (4H, s)

### Example 2

The cyclic compound shown by the following formula (V) was synthesized.

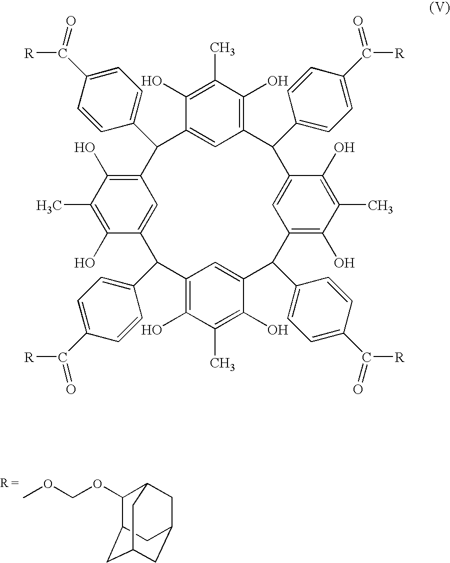

A three-neck flask (capacity: 500 ml) equipped with a thermometer, which had been replaced with a nitrogen gas, was charged with 2.51 g (2.44 mmol) of the precursor (2) synthesized in the above-mentioned Production Example 2. Then, 15 ml of DMF and 1.23 g (12.2 mmol) of triethylamine were added, followed by stirring. The flask was then cooled in an ice/water bath to allow the temperature inside of the flask to be 4° C. Then, 2.45 g (12.2 mmol) of 2-chloromethoxyadamantane was dissolved in 15 ml of DMF. The resultant was added dropwise to the flask slowly such that the temperature did not exceed 10° C. After the completion of the dropwise addition, the cooling was stopped. When the inside temperature became around room temperature, the mixture was allowed to react in a nitrogen atmosphere for 16 hours. About 250 ml of de-ionized water was poured to the reaction solution, and the resultant was further stirred for one hour. A yellowish white solid which was generated was taken out by filtration, washed with de-ionized water, and dried under vacuum.

3.63 g of the resulting solid was placed in a round-bottom flask with a capacity of 200 ml. Then, 60 ml of DMF was added, followed by heating in an oil bath of 65° C. After allowing the resultant to stand overnight, de-ionized water was gradually added to the solution inside the flask while stirring by means of a magnetic stirrer until the turbidity disappeared. After allowing the resultant to stand for one day, the generated crystals were collected by filtration, washed with a small amount of DMF, and dried under vacuum for one day, whereby 1.29 g of white crystals were obtained. As a result of ¹H-NMR, it was confirmed that the resulting compound was the calixresorcinarene derivative shown by the above-mentioned formula (V) in which R is an adamantyl-2-yl-oxymethyleneoxy group (amount: 1.29 g (0.77 mmol), yield: 32%).

The spectrum data of ¹H-NMR is given below.

¹H-NMR (tetramethylsilane as an internal standard: solvent (CD₃)₂SO: ppm): 1.45-1.48 (8H, m), 1.68-1.71 (16H, m), 1.75-1.83 (16H, m), 1.90 (6H, s), 1.98-2.04 (16H, m), 2.13 (6H, s), 3.87 (4H, s), 4.98 (2H, s), 5.54-5.58 (8H, m), 5.70 (4H, s), 6.12 (2H, s), 6.74 (8H, d), 7.45 (8H, d), 7.61 (4H, s), 7.68 (4H, s)

### Example 3

The cyclic compound shown by the following formula (VI) was synthesized.

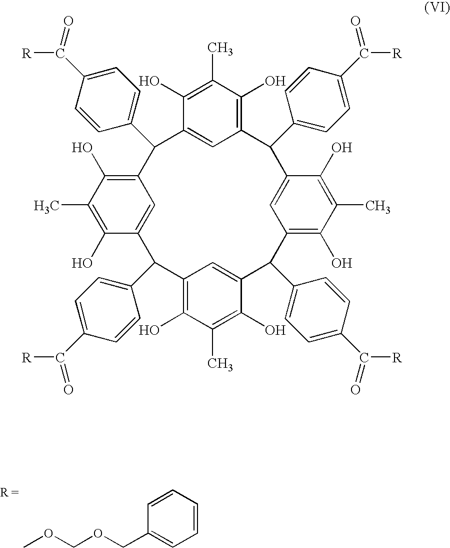

A three-neck flask (capacity: 200 ml) equipped with a thermometer, which had been replaced with a nitrogen gas, was charged with 2.0 g (1.95 mmol) of the precursor (2) synthesized in the above-mentioned Production Example 2. Then, 20 ml of DMF and 0.98 g (9.76 mmol) of triethylamine were added, followed by stirring. The flask was then cooled in an ice/water bath to allow the temperature inside of the flask to be 4° C. Then, 1.53 g (9.76 mmol, manufactured by Tokyo Chemical Industry Co., Ltd.) of benzyl chloromethyl ether was dissolved in 10 ml of DMF. The resultant was added dropwise to the flask slowly such that the temperature did not exceed 10° C. After the completion of the dropwise addition, the cooling was stopped. When the inside temperature became around room temperature, the mixture was allowed to react in a nitrogen atmosphere for 16 hours. After the completion of the reaction, the reaction mixture was diluted by pouring it to about 200 ml of de-ionized water. A yellowish white solid which was generated was extracted by dissolving with about 200 ml of ethyl acetate. The ethyl acetate layer was separated, and washed with de-ionized water and then with saturated salt solution. After drying with anhydrous sodium sulfate, the solvent was distilled off under a reduced pressure, and drying was further conducted under vacuum.

2.36 g of the resulting solid was placed in a round-bottom flask with a capacity of 100 ml. Then, 30 ml of DMF was added to allow the solid to be dissolved. Subsequently, de-ionized water was gradually added while stirring by means of a magnetic stirrer until the turbidity disappeared. After allowing the resultant to stand for one day, the generated crystals were collected by filtration, washed with a small amount of DMF, then with de-ionized water, and dried under vacuum for one day, whereby 1.05 g of white crystals were obtained. As a result of ¹H-NMR, it was confirmed that the resulting compound was the calixresorcinarene derivative shown by the above-mentioned formula (VI) in which R is a benzyloxymethyleneoxy group (amount: 1.05 g (0.70 mmol), yield: 36%).

The spectrum data of ¹H-NMR is given below.

¹H-NMR (tetramethylsilane as an internal standard: solvent (CD₃)₂SO: ppm): 1.91 (6H, s), 2.15 (6H, s), 4.73 (8H, s), 5.05 (2H, s), 5.50-5.54 (8H, m), 5.74 (4H, s), 6.17 (2H, s), 6.77 (8H, d), 7.30-7.38 (20H, m), 7.51 (8H, d), 7.67 (4H, s), 7.70 (4H, s)

### Example 4

The cyclic compound shown by the following formula (VII) was synthesized.

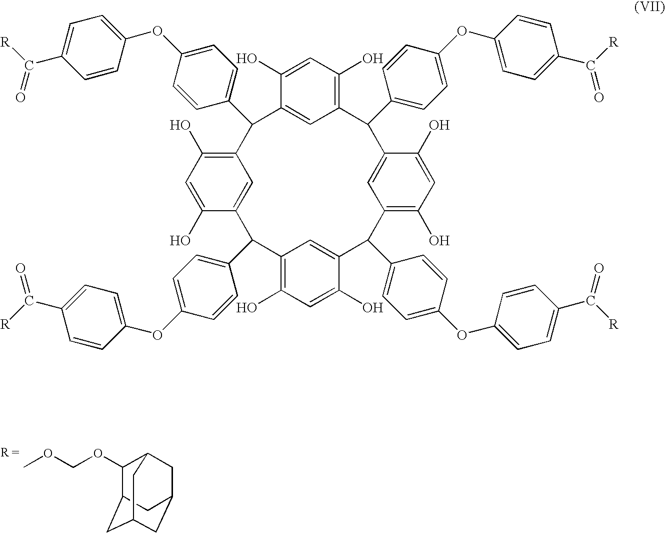

A three-neck flask (capacity: 200 ml) equipped with a thermometer, which had been replaced with a nitrogen gas, was charged with 1.2 g (0.90 mmol) of the precursor (3) synthesized in the above-mentioned Production Example 3. Then, 9 ml of DMF and 0.45 g (4.5 mmol) of triethylamine were added, followed by stirring. The flask was then cooled in an ice/water bath to allow the temperature inside of the flask to be 4° C. Then, 0.925 g (4.6 mmol) of 2-chloromethoxyadamantane was dissolved in 9 ml of DMF. The resultant was added dropwise to the flask slowly such that the temperature did not exceed 10° C. After the completion of the dropwise addition, the cooling was stopped. When the inside temperature became around room temperature, the mixture was allowed to react in a nitrogen atmosphere for 16 hours. About 100 ml of ice water was poured to the reaction solution, and the resultant was further stirred for one hour. A yellowish white solid which was generated was taken out by filtration, washed with de-ionized water, and dried under vacuum for 16 hours. 1.75 g of the resulting solid was placed in a round-bottom flask with a capacity of 100 ml. Then, 10 ml of DMF was added, followed by heating in an oil bath of 75° C. After allowing the resultant to stand for one day, de-ionized water was gradually added to the solution inside the flask while stirring by means of a magnetic stirrer until the turbidity disappeared. After allowing the resultant to stand for one day, the generated crystals were collected by filtration, washed with a small amount of DMF, and dried under vacuum for one day. As a result of ¹H-NMR, it was confirmed that the resulting compound was the cyclic compound shown by the above-mentioned formula (VII) in which R is an adamantyl-2-yl-oxymethyleneoxy group (amount: 0.90 g (0.45 mmol), yield: 50%).

The spectrum data of ¹H-NMR is given below.

¹H-NMR (tetramethylsilane as an internal standard: solvent (CD₃)₂SO: ppm): 1.40-1.43 (8H, m), 1.65-1.78 (32H, m), 1.91-1.97 (16H, m), 3.81 (4H, s), 5.53 (8H, s), 5.61 (4H, s), 5.80 (2H, s), 6.19 (2H, s), 6.35 (2H, s), 6.40 (2H, s), 6.77 (16H, dd), 6.83 (8H, d), 7.90 (8H, d), 8.60 (4H, s), 8.67 (4H, s)

### Example 5

The cyclic compound shown by the following formula (VIII) was synthesized.

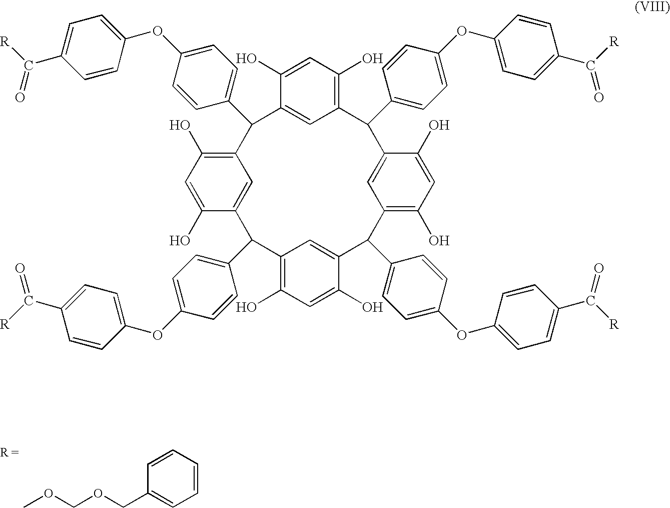

A three-neck flask (capacity: 200 ml) equipped with a thermometer, which had been replaced with a nitrogen gas, was charged with 1.20 g (0.90 mmol) of the precursor (3) synthesized in the above-mentioned Production Example 3. Then, 9 ml of DMF and 0.45 g (4.5 mmol) of triethylamine were added, followed by stirring. The flask was then cooled in an ice/water bath to allow the temperature inside of the flask to be 4° C. Then, 0.70 g (4.5 mmol, manufactured by Tokyo Chemical Industry Co., Ltd.) of benzyl chloromethyl ether was dissolved in 7 ml of DMF. The resultant was added dropwise to the flask slowly such that the temperature did not exceed 10° C. After the completion of the dropwise addition, the cooling was stopped. When the inside temperature became around room temperature, the mixture was allowed to react in a nitrogen atmosphere for 16 hours. About 100 ml of ice water was poured to the reaction solution, and the resultant was further stirred for one hour. A yellowish white solid which was generated was taken out by filtration, washed with de-ionized water, and dried under vacuum for 16 hours. As a result of ¹H-NMR, it was confirmed that the resulting compound was the cyclic compound shown by the above-mentioned formula (VIII) in which R is a benzyloxymethyleneoxy group (amount: 1.52 g (0.83 mmol), yield: 92%).

The spectrum data of ¹H-NMR is given below.

¹H-NMR (tetramethylsilane as an internal standard: solvent (CD₃)₂SO: ppm): 4.72 (8H, s), 5.53 (8H, s), 5.61 (4H, s), 5.78 (2H, s), 6.19 (2H, s), 6.35 (2H, s), 6.40 (2H, s), 6.77 (16H, dd), 6.86 (8H, d), 7.26-7.36 (20H, m), 7.91 (8H, d), 8.61 (4H, s), 8.67 (4H, s)

### Example 6

The cyclic compound shown by the following formula (IX) was synthesized.

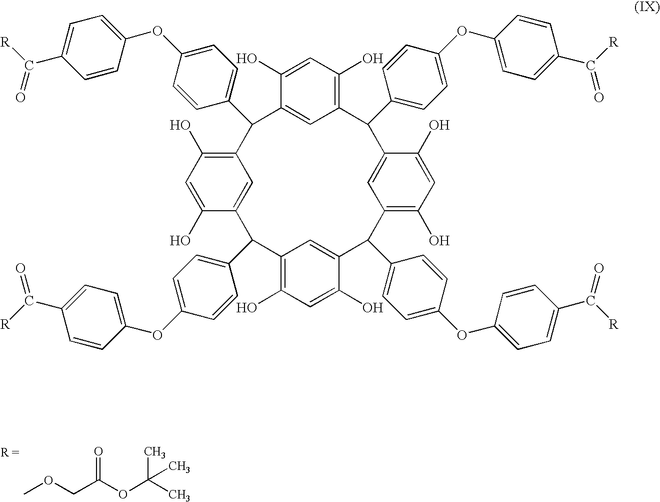

A three-neck flask (capacity: 200 ml) equipped with a thermometer, which had been replaced with a nitrogen gas, was charged with 1.0 g (0.75 mmol) of the precursor (3) synthesized in the above-mentioned Production Example 3. Then, 22 ml of DMF and 0.32 g (3.8 mmol) of sodium hydrogencarbonate were added, followed by stirring. 0.64 g of t-butyl bromoacetate (3.3 mmol, manufactured by Tokyo Chemical Industry Co., Ltd.) was dissolved in 7 ml of DMF and the resultant was added to the flask. The flask was heated in an oil bath of 65° C., thereby to allow the mixture to react for 6 hours. After the completion of the reaction, the reaction mixture was cooled to around room temperature. The reaction mixture was diluted by pouring to 150 ml of de-ionized water, and stirred for further one hour. A yellowish white solid which was generated was taken out by filtration, washed with de-ionized water, and dried under vacuum for 16 hours. As a result of ¹H-NMR, it was confirmed that the resulting compound was the cyclic compound shown by the above-mentioned formula (IX) in which R is a butoxycarbonylmethyleneoxy group (amount: 1.22 g (0.68 mmol), yield: 91%).

The spectrum data of ¹H-NMR is given below.

¹H-NMR (tetramethylsilane as an internal standard: solvent (CD₃)₂SO: ppm): 1.42 (36H, s), 4.74 (8H, s), 5.60 (4H, s), 5.60 (4H, s), 5.77 (2H, s), 6.18 (2H, s), 6.34 (2H, s), 6.39 (2H, s), 6.77 (16H, dd), 6.88 (8H, d), 7.95 (8H, d), 8.60 (4H, s), 8.67 (4H, s)

### Example 7

The cyclic compound shown by the following formula (X) was synthesized.

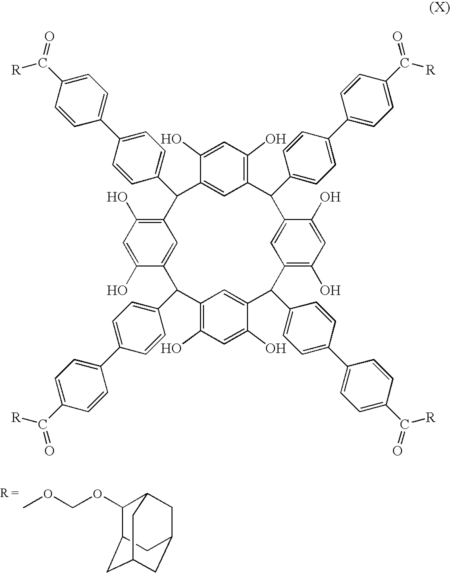

A three-neck flask (capacity: 200 ml) equipped with a thermometer, which had been replaced with a nitrogen gas, was charged with 0.80 g (0.63 mmol) of the precursor (4) synthesized in the above-mentioned Production Example 4. Then, 5 ml of DMF and 0.32 g (3.14 mmol) of triethylamine were added, followed by stirring. The flask was then cooled in an ice/water bath to allow the temperature inside of the flask to be 4° C. Then, 0.66 g (3.3 mmol) of 2-chloromethoxyadamantane was dissolved in 3 ml of DMF. The resultant was added dropwise to the flask slowly such that the temperature did not exceed 10° C. After the completion of the dropwise addition, the cooling was stopped. When the inside temperature became around room temperature, the mixture was allowed to react in a nitrogen atmosphere for 16 hours. About 100 ml of ice water was poured to the reaction solution, and the resultant was further stirred for one hour. A yellowish white solid which was generated was taken out by filtration, washed with de-ionized water, and dried under vacuum. 1.21 g of the resulting solid was placed in a round-bottom flask with a capacity of 100 ml. Then, 10 ml of DMF was added, followed by heating in an oil bath of 80° C. to allow the solid to be dissolved. After allowing the resultant to stand for one day, de-ionized water was gradually added to the solution inside the flask while stirring by means of a magnetic stirrer until the turbidity disappeared. After allowing the resultant to stand for one day, the generated crystals were collected by filtration, washed with a small amount of de-ionized water, and dried under vacuum for one day. As a result of ¹H-NMR, it was confirmed that the resulting compound was the cyclic compound shown by the above-mentioned formula (X) in which R is an adamantyl-2-yl-oxymethyleneoxy group (amount: 0.77 g (0.40 mmol), yield: 63%).

The spectrum data of ¹H-NMR is given below.

¹H-NMR (tetramethylsilane as an internal standard: solvent (CD₃)₂SO: ppm): 1.45-1.48 (8H, m), 1.68-1.83 (32H, m), 1.98-2.05 (16H, m), 3.89 (4H, s), 5.59 (8H, s), 5.63 (4H, s), 5.71 (2H, s), 6.17 (2H, s), 6.44 (4H, bs), 6.76 (8H, d), 7.21 (8H, d), 7.33 (8H, d), 7.63 (8H, d), 8.57 (4H, s), 8.73 (4H, s)

### Example 8

The cyclic compound shown by the following formula (XI) was synthesized.

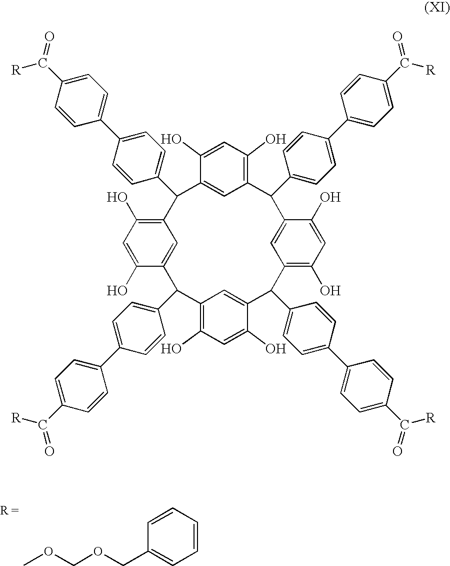

A three-neck flask (capacity: 300 ml) equipped with a thermometer, which had been replaced with a nitrogen gas, was charged with 1.54 g (1.21 mmol) of the precursor (4) synthesized in the above-mentioned Production Example 4. Then, 15 ml of DMF and 0.61 g (6.06 mmol) of triethylamine were added, followed by stirring. The flask was then cooled in an ice/water bath to allow the temperature inside of the flask to be 4° C. Then, 0.95 g (6.06 mmol, manufactured by Tokyo Chemical Industry Co., Ltd.) of benzyl chloromethyl ether was dissolved in 7 ml of DMF. The resultant was added dropwise to the flask slowly such that the temperature did not exceed 10° C. After the completion of the dropwise addition, the cooling was stopped. When the inside temperature became around room temperature, the mixture was allowed to react in a nitrogen atmosphere for 16 hours. About 150 ml of ice water was poured to the reaction solution, and the resultant was further stirred for one hour. A yellowish white solid which was generated was taken out by filtration, washed with de-ionized water, and dried under vacuum for 16 hours. It was confirmed that the resulting compound was the cyclic compound shown by the above-mentioned formula (XI) in which R is a benzyloxymethyleneoxy group (amount: 1.96 g (1.12 mmol), yield: 93%).

The spectrum data of ¹H-NMR is given below.

¹H-NMR (tetramethylsilane as an internal standard: solvent (CD₃)₂SO: ppm): 4.77 (8H, s), 5.56 (8H, s), 5.64 (4H, s), 5.73 (2H, s), 6.18 (2H, s), 6.45 (4H, bs), 6.78 (8H, d), 7.22 (8H, d), 7.28-7.41 (28H, m), 7.66 (8H, d), 8.57 (4H, s), 8.73 (4H, s)

### Example 9

The cyclic compound shown by the following formula (XII) was synthesized.

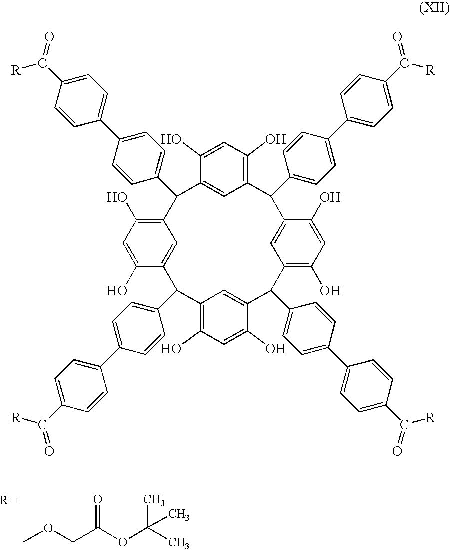

A three-neck flask (capacity: 200 ml) equipped with a thermometer, which had been replaced with a nitrogen gas, was charged with 1.27 g (1.0 mmol) of the precursor (4) synthesized in the above-mentioned Production Example 4. Then, 30 ml of DMF and 0.42 g (5.0 mmol) of sodium hydrogen carbonate were added, followed by stirring. 0.86 g of t-butyl bromoacetate (4.4 mmol, manufactured by Tokyo Chemical Industry Co., Ltd.) was dissolved in 10 ml of DMF. The resultant was added dropwise to the flask slowly such that the temperature did not exceed 20° C. After the completion of the dropwise addition, the mixture was allowed to react at room temperature in a nitrogen atmosphere for 16 hours. Then, the reaction mixture was heated to 65° C. in an oil bath, and allowed to react for further 6 hours. After the completion of the reaction, the reaction mixture was cooled to around room temperature. The reaction mixture was diluted by pouring to 150 ml of de-ionized water, and stirred for further one hour. A yellowish white solid which was generated was taken out by filtration, washed with de-ionized water, and dried under vacuum for 16 hours. As a result of ¹H-NMR, it was confirmed that the resulting compound was the cyclic compound shown by the above-mentioned formula (XII) in which R is a butoxycarbonylmethyleneoxy group (amount: 1.47 g (0.85 mmol), yield: 85%).

The spectrum data of ¹H-NMR is given below.

¹H-NMR (tetramethylsilane as an internal standard: solvent (CD₃)₂SO: ppm): 1.46 (36H, s), 4.77 (8H, s), 5.64 (4H, s), 5.74 (2H, s), 6.17 (2H, s), 6.45 (4H, bs), 6.78 (8H, d), 7.23 (8H, d), 7.37 (8H, d), 7.69 (8H, d), 8.57 (4H, s), 8.74 (4H, s)

### Comparative Example 1

A three-neck flask (capacity: 500 ml) equipped with a dripping funnel, a Dimroth condenser, and a thermometer, which had been sufficiently dried and replaced with a nitrogen gas, was charged with resorcinol (33 g, 300 mmol) and benzaldehyde (31.8 g, 300 mmol) in a nitrogen stream and sealed. Then, distilled methanol (300 ml) was added under a slight pressure of a nitrogen gas to obtain a methanol solution. The methanol solution was heated to 75° C. in an oil bath while stirring. 75 ml of concentrated hydrochloric acid was slowly added by dripping from the dripping funnel, followed by continued stirring with heating at 75° C. for two hours. After completion of the reaction, the mixture was allowed to cool to room temperature, followed by cooling in an ice water bath. The reaction mixture was allowed to stand for one hour. White crude crystals of the target compound were produced, and the crystals were filtered. These crude crystals were washed twice with purified water (100 ml), purified by recrystallization from a mixed solution of ethanol and water, and dried under reduced pressure to obtain a calixresorcinarene compound (yield: 82%).

A two-neck flask (capacity: 100 ml) equipped with a Dimroth condenser and a thermometer, which had been sufficiently dried and replaced with a nitrogen gas, was charged with the calixresorcinarene compound (3.01 g, 3.8 mmol) prepared by the above-mentioned method, sodium carbonate (3.18 g, 30 mmol), and 15-crown-5 (0.77 g, 3.18 mmol) and sealed. The flask was replaced with a nitrogen gas. Subsequently, after adding 38 ml of acetone to prepare a solution, t-butyl bromoacetate (6.82 g, 35 mmol) was added and the mixture was heated to reflux in a nitrogen atmosphere in an oil bath at 75° C. while stirring for 24 hours. Thereafter, the mixture was allowed to cool and filtered. The filtrate was allowed to reach room temperature. Ice water was poured to the reaction solution, followed by stirring for one hour to obtain a white precipitate. The precipitate was filtered and dissolved in diethyl ether (10 ml). The resulting solution was poured to an aqueous acetic acid solution (0.5 mol/l, 300 ml) to obtain white crystals. The white crystals were collected by filtration and dried under a reduced pressure, thereby to obtain a calixresorcinarene compound shown by the following formula (XIII) (amount: 2.5 g). The structure of this calixresorcinarene compound was identified by ¹H-NMR.

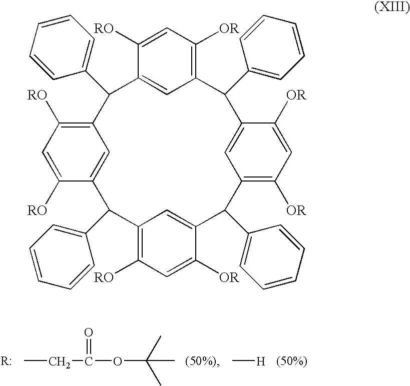

### Evaluation 1

As for the compounds synthesized in Examples 1 to 9 and Comparative Example 1, solubility was examined by using a solvent for coating which is commonly used in the field of a photoresist. The results are shown in Table 1.

TABLE 1

| | Examples | | | | | | | | | Comparative |
|---|---|---|---|---|---|---|---|---|---|---|
| | 1 | 2 | 3 | 4 | 5 | 6 | 7 | 8 | 9 | Example 1 |
| PGMEA (5 wt %) | ⊚ | ⊚ | ⊚ | ⊚ | ⊚ | ⊚ | ⊚ | ⊚ | ⊚ | X |
| PGMEA (10 wt %) | ⊚ | ⊚ | ⊚ | ⊚ | ⊚ | ⊚ | ⊚ | ⊚ | ⊚ | X |
| PGMEA (15 wt %) | ⊚ | ⊚ | ⊚ | ⊚ | ⊚ | ⊚ | ⊚ | ⊚ | ⊚ | X |
| Cyclohexanone (5 wt %) | ⊚ | ⊚ | ⊚ | ⊚ | ⊚ | ⊚ | ⊚ | ⊚ | ⊚ | ⊚ |
| Cyclohexanone (10 wt %) | ⊚ | ⊚ | ⊚ | ⊚ | ⊚ | ⊚ | ⊚ | ⊚ | ⊚ | ◯ |
| Cyclohexanone (15 wt %) | ⊚ | ⊚ | ⊚ | ⊚ | ⊚ | ⊚ | ⊚ | ⊚ | ⊚ | ◯ |

PGMEA: propylene glycol methyl ether acetate

The results of the solubility test were evaluated as follows:

⊚: Easily dissolved (solubility of a degree that the solution becomes a transparent solution by visual observation at room temperature without heating).
○: Dissolved by heating at 50° C. (solubility of a degree that the solution becomes a transparent liquid by visual observation by heating the liquid to 50° C.).
x: Not soluble (solubility of a degree that solid matters remain but the liquid is colored by visual observation at room temperature).

### Evaluation 2

A photoresist solution was prepared, and a pattern was formed on a silicon wafer using an electron beam and EUV.

87 parts by weight of the compound (VIII) synthesized in Example 5 was used as the base material, 10 parts by weight of triphenylsulfonium trifluoromethansulfonate was used as a PAG and 3 parts by weight of 1,4-diazabicyclo[2.2.2]octane was used as a quencher. A photoresist solution was produced by dissolving these solid components in propylene glycol methyl ether acetate such that the concentration of these components became 5 wt %.

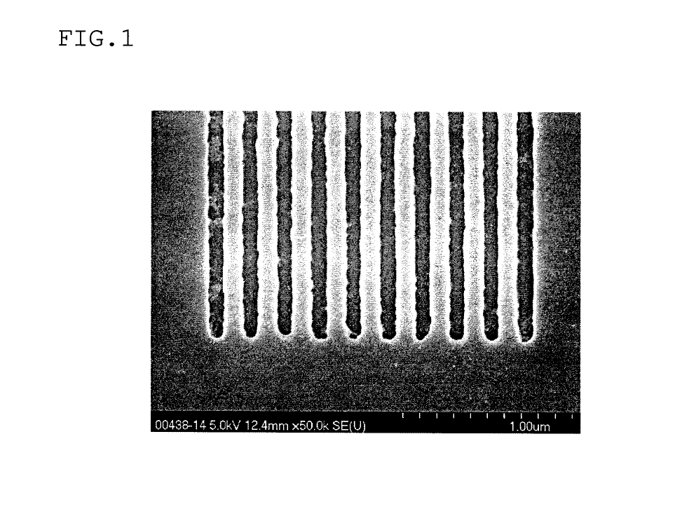

This photoresist solution was applied onto a silicon wafer which had been subjected to a HMDS treatment by spin coating and heated at 100° C. for 180 seconds to form a thin film. The substrate with the thin film was subjected to lithography by using an electron beam lithography apparatus (accelerated voltage: 50 kV). After baking at 100° C. for 60 seconds, the substrate was developed in a 2.38 wt % aqueous tetrabutylammonium hydroxide solution for 60 seconds, and washed with purified water for 60 seconds, followed by drying with a nitrogen gas stream. As a result, a line-and-space pattern of 100 nm as shown in FIG. 1 could be obtained.

By using a EUV exposure apparatus instead of the electron lithography apparatus, the substrate with the above-mentioned photoresist thin film was then exposed to EUV (wavelength: 13. 5 nm). Thereafter, the substrate was baked at 100° C. for 90 seconds, and rinsed with a 2.38 wt % aqueous tetramethylammonium hydroxide solution for 30 seconds, and then with ion-exchange water for 30 seconds to form a pattern. As a result of an observation by means of a scanning electron microscope, a line-and-space pattern similar to that obtained by means of the electron beam lithography apparatus was confirmed.

### Example 10

The cyclic compound shown by the following formula (XIV) was obtained in the same manner as in Example 6, except that the precursor (1) synthesized in Production Example 1 was used instead of the precursor (3).

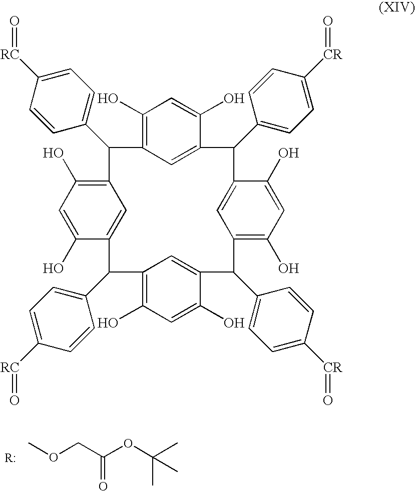

As a result of ¹H-NMR, it was confirmed that the compound was the cyclic compound shown by the above-mentioned formula (XIV) (yield: 65%).

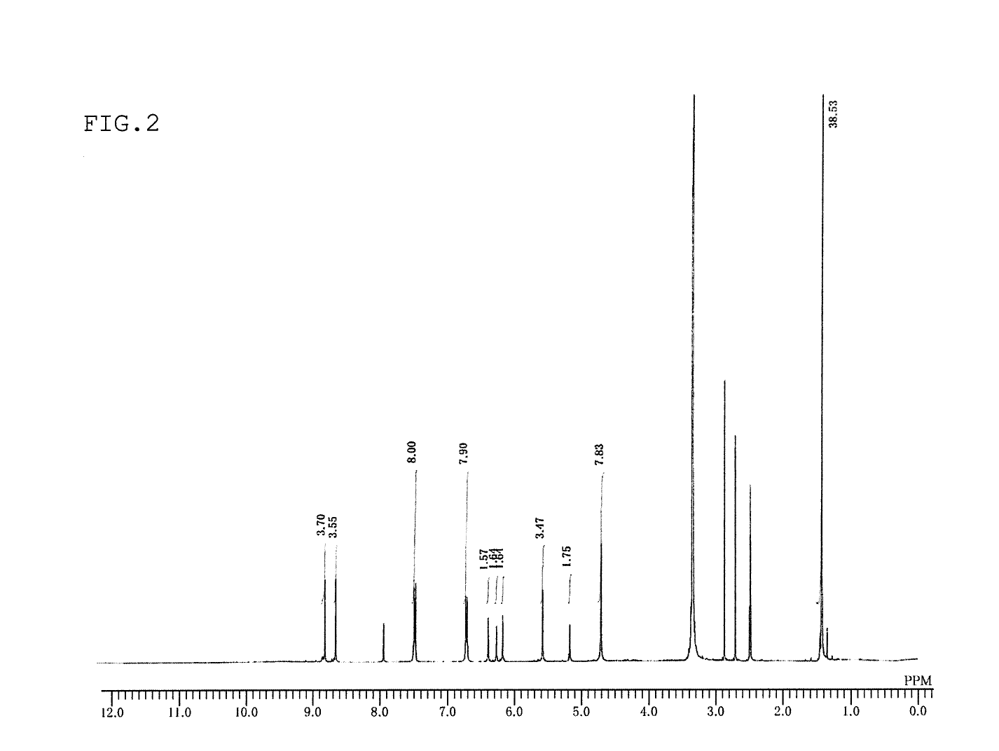

FIG. 2 shows a ¹H-NMR spectrum (solvent: DMSO).

### Example 11

The cyclic compound shown by the following formula (XV) was obtained in the same manner as in Example 6, except that the precursor (1) synthesized in Production Example 1 was used instead of the precursor (3) and 2-methyl-2-adamantyl bromoacetate was used instead of t-butyl bromoacetate.

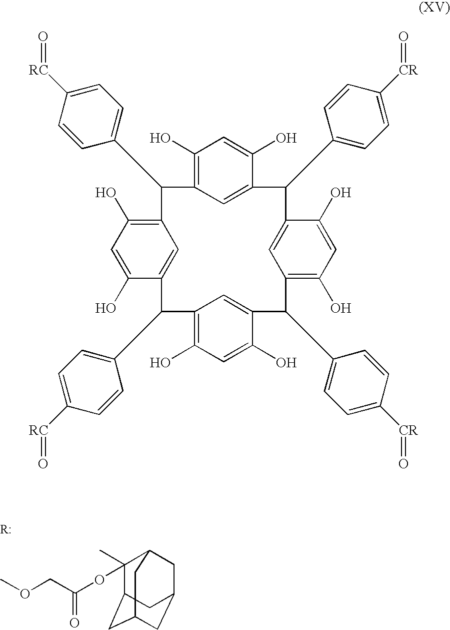

As a result of ¹H-NMR, it was confirmed that the compound was the cyclic compound shown by the above-mentioned formula (XV) (yield: 62%).

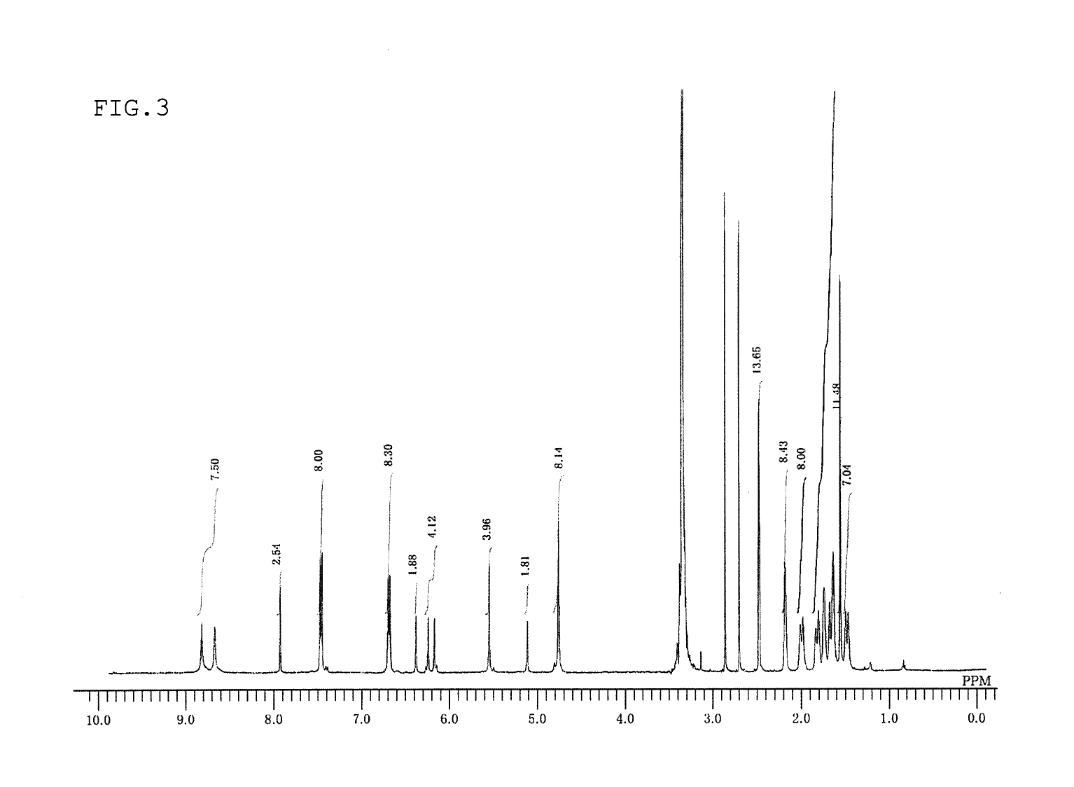

FIG. 3 shows a ¹H-NMR spectrum.

### Example 12

The cyclic compound shown by the following formula (XVI) was obtained in the same manner as in Example 3, except that the precursor (1) synthesized in Production Example 1 was used instead of the precursor (2).

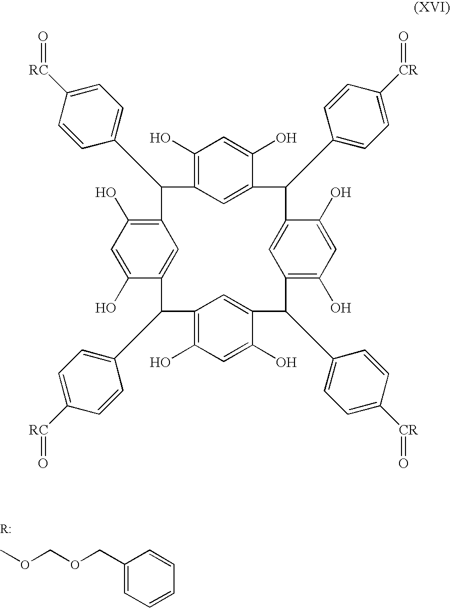

As a result of ¹H-NMR, it was confirmed that the compound was the cyclic compound shown by the above-mentioned formula (XVI) (yield: 61%).

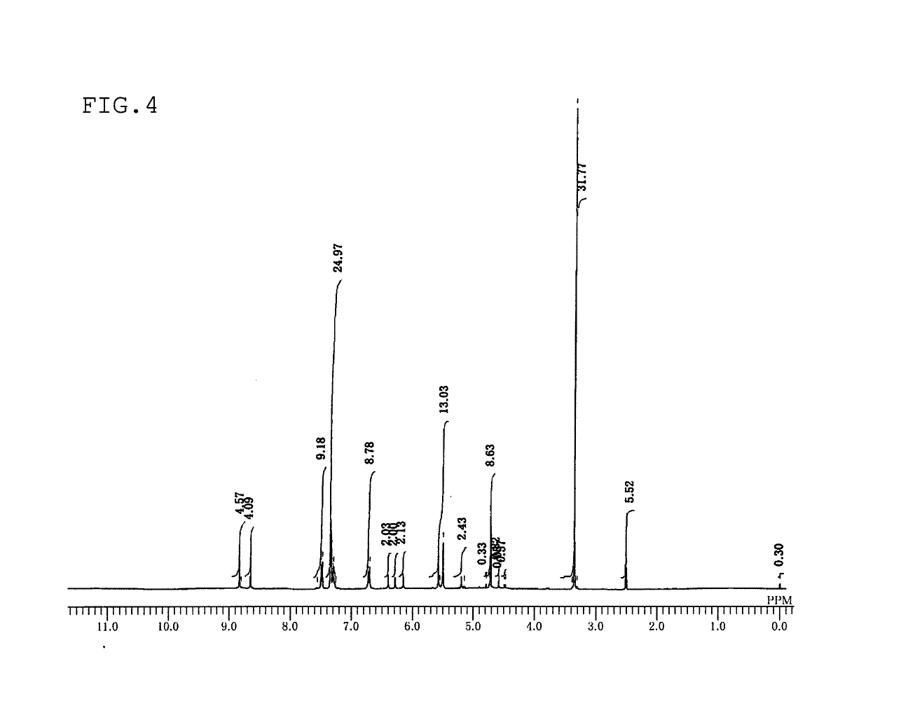

FIG. 4 shows a ¹H-NMR spectrum.

### Example 13

The cyclic compound shown by the following formula (XVII was obtained in the same manner as in Example 6 except that the precursor (2) synthesized in Production Example 2 was used instead of the precursor (3).

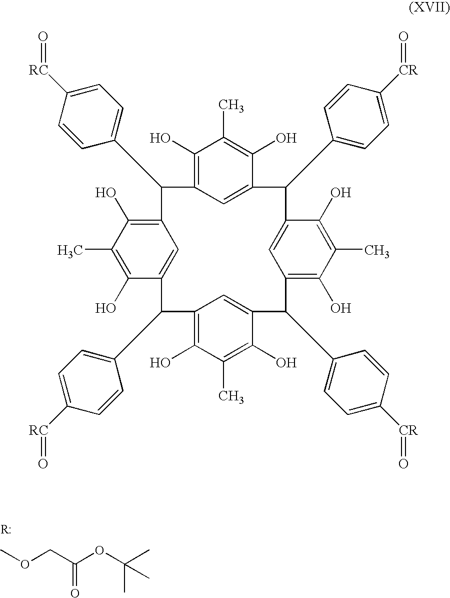

As a result of ¹H-NMR, it was confirmed that the compound was the cyclic compound shown by the above-mentioned formula (XVII) (yield: 60%).

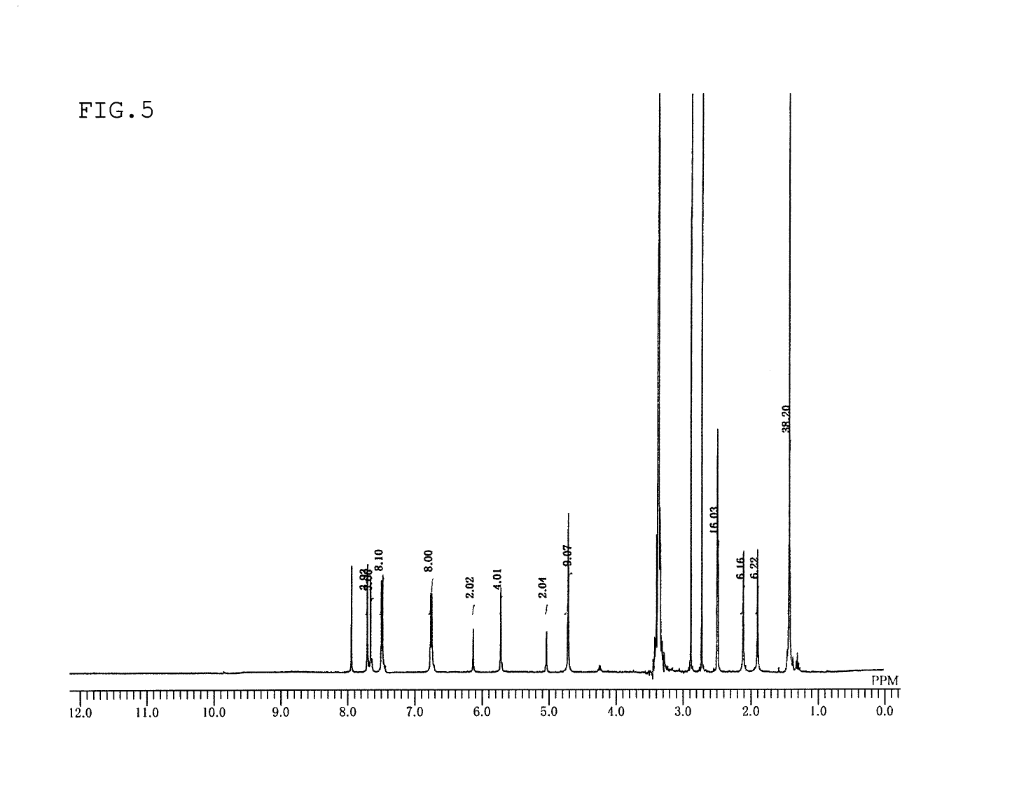

FIG. 5 shows a ¹H-NMR spectrum.

### Example 14

The cyclic compound shown by the following formula (XVIII) was obtained in the same manner as in Example 6 except that 2-methyl-2-adamantyl bromoacetate was used instead of t-butyl bromoacetate.

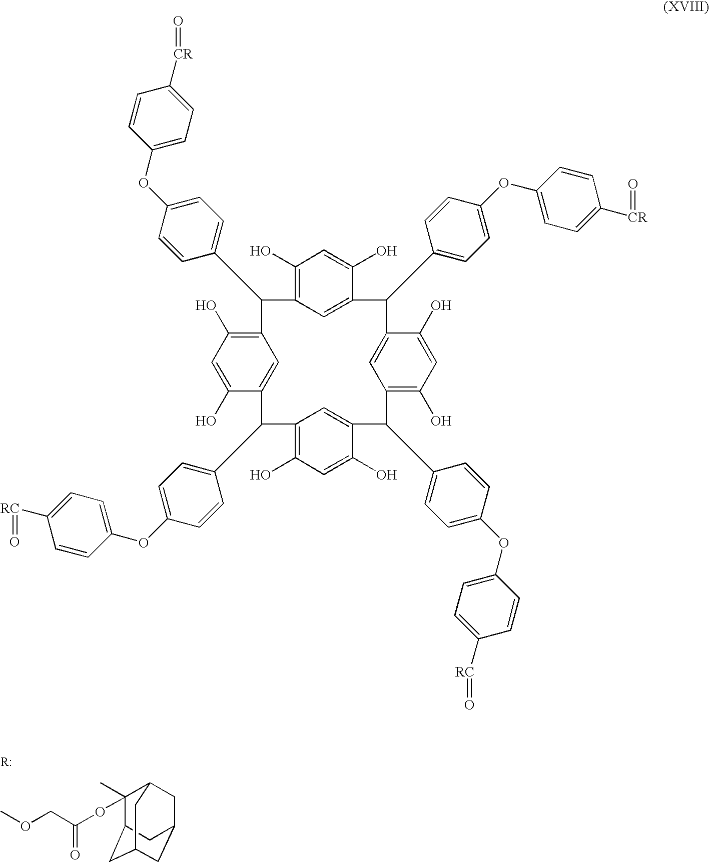

As a result of ¹H-NMR, it was confirmed that the compound was the cyclic compound shown by the above-mentioned formula (XVIII) (yield: 76%).

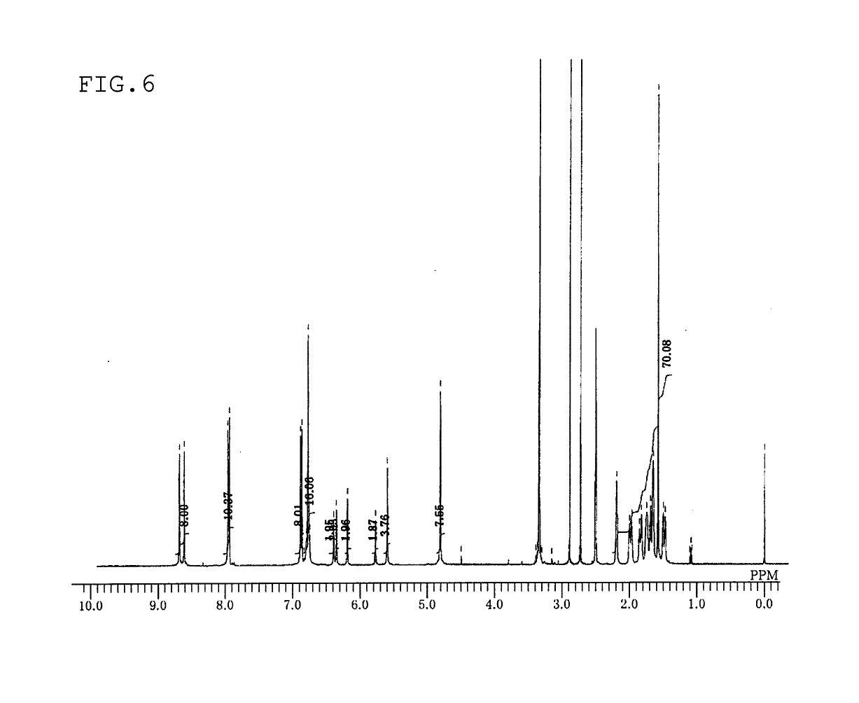

FIG. 6 shows a ¹H-NMR spectrum.

### INDUSTRIAL APPLICABILITY

The photoresist base material and the composition thereof of the invention can be preferably used in the fields of electricity and electronics such as a semiconductor device, the optical field, and other fields. This photoresist base material and the composition thereof can remarkably improve the performance of semiconductor devices such as an ULSI.

1. A cyclic compound shown by the following formula (I): 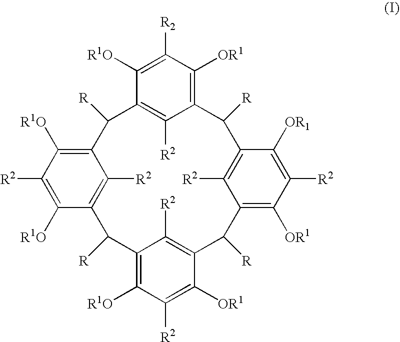
wherein R is a group shown by the following formula (1);
R¹s are independently hydrogen, a substituted or unsubstituted straight-chain aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group, or a group having a structure in which these groups and a divalent group selected from a substituted or unsubstituted alkylene group, a substituted or unsubstituted arylene group, a substituted or unsubstituted silylene group, a group formed by bonding two or more of these groups or a group formed by combining one or more of these groups and one or more of an ester group, a carbonic ester group and an ether group are bonded;
R²s are independently hydrogen, a group shown by —O—R¹, a straight-chain aliphatic hydrocarbon group having 1 to 20 carbon atoms, a branched aliphatic hydrocarbon group having 3 to 12 carbon atoms, a cyclic aliphatic hydrocarbon group having 3 to 20 carbon atoms, an aromatic group having 6 to 10 carbon atoms or a group containing an oxygen atom; and
plural Rs, R¹s and R²s in the formula (I) may be the same or different: 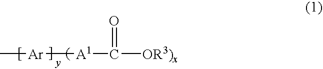
wherein Ar is an arylene group having 6 to 10 carbon atoms, a group formed by combining two or more arylene groups having 6 to 10 carbon atoms, or a group formed by combining one or more of an arylene group having 6 to 10 carbon atoms and at least one of an alkylene group and an ether group;
A¹ is a single bond, an alkylene group, an ether group or a group formed by combining two or more of an alkylene group and an ether group;
R³s are independently hydrogen, a substituted or unsubstituted straight-chain aliphatic hydrocarbon group having 1 to 20 carbon atoms, a substituted or unsubstituted branched aliphatic hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group, or a group having a structure in which these groups and a divalent group selected from a substituted or unsubstituted alkylene group, a substituted or unsubstituted arylene group, a substituted or unsubstituted silylene group, a group formed by bonding two or more of these groups or a group formed by combining one or more of these groups and one or more of an ester group, a carbonic ester group and an ether group are bonded;
x is an integer of 1 to 5;
y is an integer of 0 to 3; and
plural R³s, Ars, A¹s, xs and ys may be the same or different, providing that a case where all of R¹ and R² are hydrogen and R is a 4-carboxyphenyl group or a 4-(carboxylmethyleneoxy)phenyl group is excluded.

2. The cyclic compound according to claim 1, wherein the group shown by the formula (1) is any of groups shown by the following formulas (1-1) to (1-6): 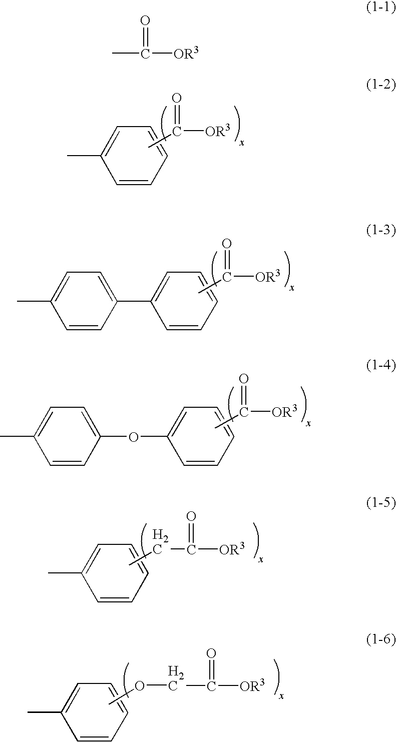
wherein R³s are independently hydrogen, a substituted or unsubstituted straight-chain aliphatic hydrocarbon group having 1 to 20 carbon atoms, a substituted or unsubstituted branched hydrocarbon group having 3 to 12 carbon atoms, a substituted or unsubstituted cyclic aliphatic hydrocarbon group having 3 to 20 carbon atoms, a substituted or unsubstituted aromatic group having 6 to 10 carbon atoms, an alkoxyalkyl group, a silyl group or a group having a structure in which these groups and a divalent group selected from a substituted or unsubstituted alkylene group, a substituted or unsubstituted arylene group, a substituted or unsubstituted silylene group, a group formed by bonding two or more of these groups or a group formed by combining one or more of these groups and one or more of an ester group, a carbonic ester group and an ether group are bonded; and
x is an integer of 1 to 5.

3. The cyclic compound according to claim 1, wherein at least one of the R¹s or at least one of the R³s is an acid-dissociative dissolution inhibiting group, and the acid-dissociative dissolution inhibiting group is a group selected from an alkoxycarbonyl group, an alkoxycarbonylmethyl group, an alkoxymethyl group, an alkoxyalkylmethyl group, an alkoxyarylmethyl group, an alkoxycarbonylphenyl group, a bis(alkoxycarbony)phenyl group and a tris(alkoxycarbonyl)phenyl group.

4. The cyclic compound according to claim 3, wherein the acid-dissociative dissolution inhibiting group is a group selected from groups shown by the following formulas (2) to (17): 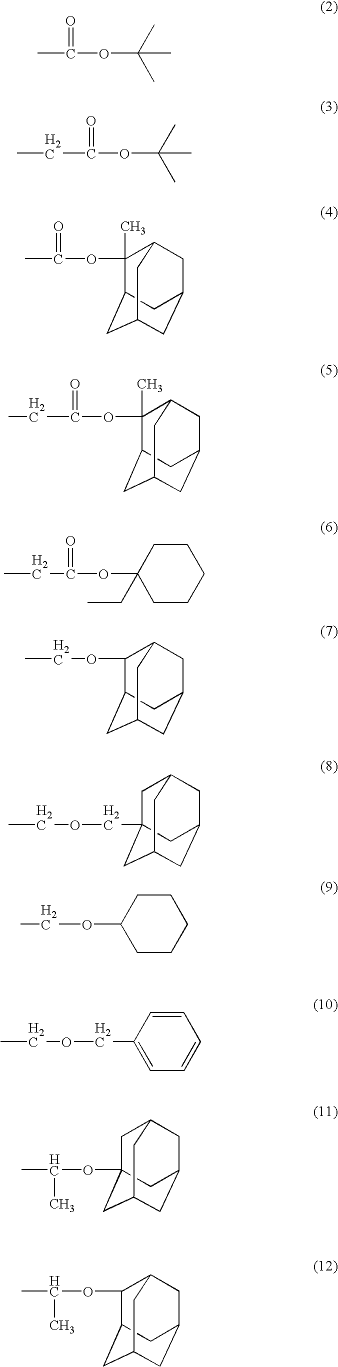 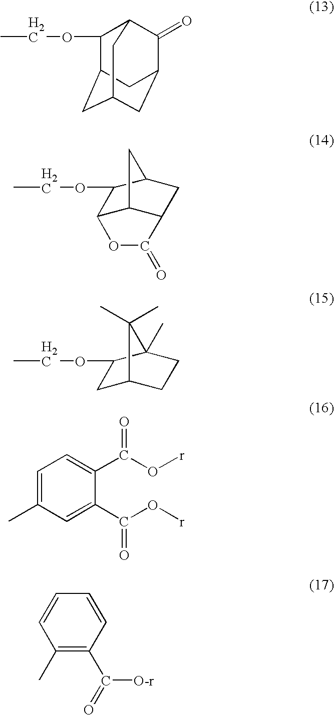
wherein r in the formulas (16) and (17) is a monovalent group selected from groups shown by the above formulas (2) to (15) and the following formulas (18) to (20):

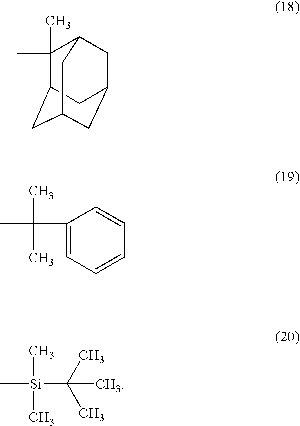

5. A photoresist base material comprising the cyclic compound according to claim 1.

6. A photoresist composition comprising the photoresist base material according to claim 5 and a solvent.

7. The photoresist composition according to claim 6 which further comprises a photoacid generator.

8. The photoresist composition according to claim 6 which further comprises a basic organic compound as a quencher.

9. A fine processing method using the photoresist composition according to claim 6.

10. A semiconductor apparatus which is produced by the fine processing method according to claim 9.
